(19) United States
(12) Patent Application Publication
(10) Pub. No.: US 2014/0274794 A1
(43) Pub. Date: Sep. 18, 2014

(54) Methods and Compositions for Diagnosis of Ovarian Cancer

(71) Applicants: David W. Speicher, Berwyn, PA (US); Hsin-Yao Tang, Media, PA (US); Lynn A. Beer, Medford, NJ (US)

(72) Inventors: David W. Speicher, Berwyn, PA (US); Hsin-Yao Tang, Media, PA (US); Lynn A. Beer, Medford, NJ (US)

(73) Assignee: THE WISTAR INSTITUTE OF ANATOMY AND BIOLOGY, Philadelphia, PA (US)

(21) Appl. No.: 14/342,646

(22) PCT Filed: Sep. 7, 2012
(86) PCT No.: PCT/US12/54136

§ 371 Date: May 1, 2014

Related U.S. Application Data
(60) Provisional application No. 61/532,881, filed on Sep. 9, 2011.

(51) Int. Cl. C12Q 1/68; G01N 33/574

(52) U.S. Cl. CPC C12Q 1/6886; G01N 33/57449
USPC 506/9; 530/389.1; 506/18; 436/501; 435/7.92; 536/24.31; 506/16; 435/6.11; 435/23

(57) ABSTRACT

Methods and compositions are provided for diagnosing ovarian cancer in a mammalian subject, preferably in a serum or plasma sample of a human subject. The methods and compositions enable the detection or measurement in the sample or from a protein level profile generated from the sample, the protein level of one or more specified biomarkers. Comparing the protein level(s) of the biomarker(s) in the subject's sample or from protein abundance profile of multiple biomarkers, with the level of the same biomarker(s) or profile in a reference standard, permits the determination of a diagnosis of ovarian cancer, or the identification of a risk of developing ovarian cancer, or enables the monitoring of the status of progression or remission of ovarian cancer in the subject followed during a therapeutic protocol.

### STATEMENT REGARDING FEDERALLY SPONSORED RESEARCH OR DEVELOPMENT

This invention was made with government support under Grant Nos. CA131582, CAl20393 and CA10815 awarded by the National Institutes of Health. The government has certain rights in this invention.

### BACKGROUND OF THE INVENTION

Ovarian cancer is the fifth-leading cause of cancer-related death in women in the United States, and is the most lethal of all gynecological malignancies.¹ In 2010, an estimated 21,880 women were diagnosed with ovarian cancer, and 13,850 deaths occurred in the United States alone.¹ The most common and deadly form of ovarian cancer is epithelial ovarian cancer, which further can be divided into four major histopathological groups: serous, endometrioid, mucinous and clear cell tumors.^{2, 3} The high mortality rate of ovarian cancer is due largely to the lack of effective screening strategies for early detection. When ovarian cancer is diagnosed at an early stage (stages I or II), treatment is highly effective, with a five-year survival rate of up to 90%, whereas the five-year survival rate for patients with advanced disease (stages III and IV) is reduced to 30% or less.^{4, 5} Unfortunately, most ovarian cancers are not diagnosed until after the cancer has spread, primarily because earlier-stage diseases are asymptomatic and the ovaries are buried deep within the body.

Current screening methods for ovarian cancer typically use a combination of pelvic examination, transvaginal ultrasonography, and serum CA125, but these methods are not effective in detecting early-stage ovarian cancer.⁶⁻⁸ In addition, CA125 is recognized as a poor protein biomarker for early detection due to its high false positive rate and poor sensitivity and specificity.^{9, 10} Other promising biomarkers have been reported,^{11, 12} but a recently completed study comparing many of these protein biomarkers showed that none of them performed better than CA125 as a biomarker for ovarian cancer.¹³ A few groups also have used panels of biomarkers and obtained better sensitivity and specificity than CA125 alone when used in diagnostic samples.¹⁴⁻¹⁷ However, a recent study found that available biomarker panels did not outperform CA125 when used in prediagnostic samples.¹⁸

### SUMMARY OF THE INVENTION

In one aspect, a diagnostic reagent or device includes at least one ligand capable of specifically complexing with, binding to, quantitatively detecting, or identifying a single target protein biomarker of Table 1, or a physiological molecular form, modified molecular form, isoform, pro-form, or peptide fragment thereof. In one embodiment, at least one ligand is associated with a detectable label or with a substrate. In another embodiment, the reagent comprises multiple ligands, each ligand directed to a different biomarker. In another embodiment, the reagent additionally contains at least one ligand that specifically complexes with, binds to, quantitatively detects, or identifies an additional known ovarian cancer biomarker, e.g., CA125, or a molecular form, isoform, pro-form, modified molecular form, or peptide fragment therefrom.

In another aspect the reagents are selected from an antibody or fragment of an antibody, antibody mimic, or equivalent that binds to or complexes with a biomarker of Table 1, or a molecular form, modified molecular form, isoform, pro-form, or peptide fragment thereof. In certain embodiments, these antibodies or fragments are associated with a detectable label or immobilized on a suitable substrate.

In another aspect, a diagnostic device include a kit, panel or microarray comprising at least two diagnostic reagents, each reagent identifying a different biomarker of Table 1, or molecular form, modified molecular form, isoform, pro-form, or peptide fragment thereof. In another embodiment, the kit, panel or microarray includes diagnostic reagents that bind or complex individually to 2, 3, 4, 5, 6, 7, 8, 9, 10 and 11 or more different biomarkers, molecular forms or fragments thereof, desirably in a protein level profile or panel. In another embodiment, the kit, panel or microarray includes diagnostic reagents that bind or complex to different molecular forms or fragments of the same biomarker, desirably in a protein level profile or panel.

In another aspect, a method for diagnosing or detecting the existence or absence of, or monitoring the progress of, ovarian cancer in a subject is provided. This method includes contacting a sample obtained from a test subject with a composition or reagent as described herein; and detecting or measuring in the sample or from a protein level profile generated from the sample, the protein levels of one or more of the biomarkers of Table 1, or a molecular form, modified molecular form, isoform, pro-form, or peptide fragment thereof. The protein level(s) of the biomarker(s) in the subject's sample or from a protein level profile or ratio of multiple biomarkers is then compared with the level or ratio of the same biomarker or biomarkers in a reference standard. A significant change in protein level or ratio of the subject's sample biomarker or biomarkers from that in the reference standard indicates a diagnosis, risk, or the status of progression or remission of ovarian cancer in the subject.

In certain embodiments, the method and the interpretation of the end result differs depending the reference standard, which is generated from a reference human subject or a population of subjects have having certain conditions as described below. For example, in one embodiment, the reference standard is generated from healthy subjects with no ovarian cancer. In another embodiment, a reference standard is generated from a human subject or a population of subjects having ovarian cancer or a subtype, as detailed below. In certain embodiments, the method differs based upon the type, condition and timing of the subject's sample being tested. For example, the sample may be serum or plasma. Depending upon the selection of the subject's samples and the reference standard and the relationship thereof, the change in protein level of each biomarker may involve an increase in comparison to the reference or a decrease in comparison to the reference.

In still other aspects, optional labels, label systems, substrates for immobilization and controls may be included in or with the reagent or kit, and used in these diagnostic methods to identify a characteristic change in the protein level or protein abundance level of the one or more biomarker indicative of the diagnosis of ovarian cancer.

In another aspect, a method for diagnosing or detecting or monitoring the progress of ovarian cancer in a subject employs non-ligand assays, such as mass spectrometry or liquid chromatography/mass spectrometry.

In another aspect, use of any of the diagnostic reagents described herein in a method for the diagnosis of ovarian cancer is provided.

Other aspects and advantages of these compositions and methods are described further in the following detailed description of the preferred embodiments thereof.

### BRIEF DESCRIPTION OF THE DRAWINGS

FIG. 1A is a flowchart showing an overview of the experimental workflow for ovarian cancer biomarker discovery. Sera from xenograft SCID mice harboring TOV-112D ovarian cancer cells were subjected to four-dimensional separation prior to LC-MS/MS analysis on an LTQ-FT mass spectrometer.

FIG. 1B is a flowchart showing an overview of the experimental workflow for ovarian cancer verification/validation. Control and cancer patient sera were subjected to three-dimensional separation prior to quantitative LC-MRM analysis on a 4000 QTRAP mass spectrometer.

FIG. 2 is a bar graph showing non-redundant proteins identified in the ovarian TOV-112D xenograft mouse serum proteome. The bar chart shows the number of unique mouse proteins (Mouse), proteins where all identified peptides were common to homologous mouse and human proteins (Indistinguishable), and unique human proteins (Human). The distribution of proteins identified by one unique peptide (hatched bar), two unique peptides (white bar), and greater than two unique peptides (black bar). The total number of proteins identified by more than one unique peptide is indicated on top of each bar.

FIG. 3A is an MS/MS spectrum of a human-specific peptide cathepsin D (CTSD) identified from the ovarian TOV-112D xenograft mouse serum. Sequence alignment of human peptide (Hu) K.FDGILGMAYPR.I SEQ ID NO:16 with its corresponding mouse peptide (Mo) K.FDGILGMGYPHI SEQ ID NO:17 is shown, and the protein sequence homology between the two species is indicated. In this figure and throughout this specification and other figures, a period in the sequence represents a site of trypsin cleavage. The sequence between the periods is the actual tryptic peptide sequence identified from MS and MS/MS data. The periods define the peptide boundaries. Amino acid residues before the peptide and after the peptide are the flanking residues in the matched protein sequence.

FIG. 3B is an MS/MS spectrum of a human-specific peptide clusterin (CLU) identified from the ovarian TOV-112D xenograft mouse serum. Sequence alignment of human peptide (Hu) K.LFDSDPITVTVPVEVSR.K SEQ ID NO:18 with its corresponding mouse peptide (Mo) K.LFDSDPITVVLPEEVSK.D SEQ ID NO:19 is shown, and the protein sequence homology between the two species is indicated.

FIG. 4 is a sequence alignment of the human CLIC1 protein SEQ ID NO: 1 (top line) and mouse CLIC1 protein SEQ ID NO: 2 (bottom line). The identified CLIC1 peptides (identified common peptides in a clear box; identified human peptides in a shaded box) are indicated. Tryptic sites (K or R) are indicated in bold. Species differences are indicated by lowercase, bold.

FIG. 5A is a GeLC-MRM quantitation of CLIC1 in pooled serum samples from patients with benign (n=9) and advanced (stage III, n=6; stage IV, n=3) ovarian cancer. The unfilled column represents the sequence LAALNPESNTAGLDIFAK (amino acids 96-113 of SEQ ID NO: 1; the darker shaded column represents GVTFNVTTVDTK (amino acids 38-49 of SEQ ID NO:1); the lighter shaded column represents the sequence NSNPALNDNLEK (amino acid 120-131 of SEQ ID NO: 1); the black column represents the average. The MRM transitions used for quantitation are indicated.

FIG. 5B is a GeLC-MRM quantitation of CTSD-30 kDa in pooled serum samples from patients with benign (n=9) and advanced (stage III, n=6; stage IV, n=3) ovarian cancer. The MRM transitions used for quantitation are indicated. The unfilled column represents the sequence QVFGEATK SEQ ID NO: 3; the gray column represents sequence VGFAEAAR SEQ ID NO: 4; and the black column represents the average.

FIG. 5C is a GeLC-MRM quantitation of CLU in pooled serum samples from patients with benign (n=9) and advanced (stage III, n=6; stage IV, n=3) ovarian cancer. Asterisk indicates a CLU peptide affected by spray instability, and was not used in computing the Average value. The unfilled column represents the sequence SGSGLVGR SEQ ID NO: 5; the darker gray column represents the sequence ASSIIDELFQDR SEQ ID NO: 6; the lighter gray column represents the sequence VTTVASHTSDSDVPSGVTEVVVK SEQ ID NO: 7; and the black column represents the average. The MRM transitions used for quantitation are indicated.

FIG. 6A is a scatter plot showing LC-MRM quantitation of CLIC1 in serum of ovarian cancer patients (Cancer: 15 stage III, 3 stage IV) and individuals without ovarian cancer (Control: 6 normal, 9 benign).

FIG. 6B is a scatter plot of the same data comparing Normal and Benign groups separately with the Cancer samples. P-values were calculated using Student's t-test. Horizontal bars in each dataset indicate the average serum level of the protein. MRM transitions used and quantitation values for all samples are listed in Tables 4A through 4C.

FIG. 6C is a scatter plot showing LC-MRM quantitation of CTSD-30 kDa in serum of ovarian cancer patients (Cancer: 15 stage III, 3 stage IV) and individuals without ovarian cancer (Control: 6 normal, 9 benign).

FIG. 6D is a scatter plot of the same data comparing Normal and Benign groups separately with the Cancer samples. P-values were calculated using Student's t-test. Horizontal bars in each dataset indicate the average serum level of the protein. MRM transitions used and quantitation values for all samples are listed in Tables 4A through 4C.

FIG. 6E is a scatter plot showing LC-MRM quantitation of PRDX6 in serum of ovarian cancer patients (Cancer: 15 stage III, 3 stage IV) and individuals without ovarian cancer (Control: 6 normal, 9 benign).

FIG. 6F is a scatter plot of the same data comparing Normal and Benign groups separately with the Cancer samples. P-values were calculated using Student's t-test. Horizontal bars in each dataset indicate the average serum level of the protein. MRM transitions used and quantitation values for all samples are listed in Tables 4A through 4C.

FIG. 7A is an ROC curve of CLIC1. ROC curves were generated from Control (6 normal, 9 benign) and Cancer (15 stage III, 3 stage IV) datasets. The area under the ROC curve (AUC) is indicated.

FIG. 7B is an ROC curve of CTSD-30 kDa. ROC curves were generated from Control (6 normal, 9 benign) and Cancer (15 stage III, 3 stage IV) datasets. The area under the ROC curve (AUC) is indicated.

FIG. 7C is an ROC curve of PRDX6. ROC curves were generated from Control (6 normal, 9 benign) and Cancer (15 stage III, 3 stage IV) datasets. The area under the ROC curve (AUC) is indicated.

FIG. 8 is a graph showing a comparison of GeLC-MRM quantitation of CTSD-30 kDa, CLIC1, and PRDX6 from pooled samples versus average of individual samples. White bars, GeLC-MRM quantitation obtained from a pool of Normal (n=6) and a pool of Cancer (n=9) serum samples. Black bars, GeLC-MRM quantitation obtained from the six individual Normal (average) and nine individual Cancer (average) serum samples.

FIG. 9A is a GeLC-MRM quantitation of TPM1 in pooled serum samples from patients with normal/benign and advanced ovarian cancer. The white column represents the sequence SLQEQADAAEER SEQ ID NO: 8; the gray column represents YEEEIK SEQ ID NO: 9; the black column represents the average.

FIG. 9B is a scatter plot showing LC-MRM quantitation of TPM1 protein intensity in serum of ovarian cancer patients and individuals without ovarian cancer (both normal and benign groups). P-values were calculated using Student's t-test. Horizontal bars in each dataset indicate the average serum level of the protein.

FIG. 9C is a scatter plot showing protein intensity of TPM1 for the same data as in FIG. 9B but plotted on a log scale.

FIG. 9D is an updated scatter plot performed on more patient samples showing LC-MRM quantitation of TPM1, isoform 6 (UniProt Ref Q1ZYL5), protein intensity in serum of ovarian cancer patients and individuals without ovarian cancer (both normal and benign groups). P-value 0.0052 was calculated using the Mann-Whitney test. Horizontal bars in each dataset indicate the average serum level of the protein.

FIG. 10A is a scatter plot showing LC-MRM quantitation of PSMA7 in serum of ovarian cancer patients and individuals without ovarian cancer (normal and benign, separately). P-values were calculated using a standard statistical test, e.g., the Mann-Whitney test. Horizontal bars in each dataset indicate the average serum level of the protein. MRM transitions used and quantitation values for all samples are listed in Tables 4A through 4C.

FIG. 10B is a scatter plot showing LC-MRM quantitation of BPGM in serum of ovarian cancer patients and individuals without ovarian cancer (normal and benign, separately). P-values were calculated a standard statistical test, e.g., the Mann-Whitney test. Horizontal bars in each dataset indicate the average serum level of the protein. MRM transitions used and quantitation values for all samples are listed in Tables 4A through 4C.

### DETAILED DESCRIPTION OF THE INVENTION

The compositions and methods described herein provide means for diagnosing or detecting the existence or absence of, or monitoring the progress of, ovarian cancer in a subject using one or more of the biomarkers identified in Table 1 in optional combination with one or more known ovarian cancer-associated biomarkers. As demonstrated in the examples below, the inventors combined a xenograft mouse model using the ovarian endometrioid TOV-112D cell line with a higher performance, multidimensional prefractionation strategy to identify human proteins in the mouse serum. Identified biomarkers are shed by the tumor into the blood. In one embodiment, low-abundance human proteins are present at less than about 100 ng/ml in normal human serum. In other embodiments, low-abundance human proteins are present at less than about 80, 50, 30, 20, 10 or 1 ng/ml in normal human serum. Such difficult-to-detect proteins, which are in lower abundance in the serum, are more tumor-specific than proteins present in greater concentrations in the serum.

Because certain biomarkers identified in Table 1 were found by the inventors to be secreted in low abundance into serum from the tumor tissue, the compositions and methods described herein involve detection and/or quantitative evaluation of these biomarkers, individually or in combination with the other biomarkers in Table 1 and/or with additional known ovarian cancer biomarkers in a sample, desirably a serum/plasma or blood sample, from a subject. Such detection and evaluation permits diagnosis of ovarian cancer in a less invasive manner than is currently available using a tissue biopsy or a surgical sample. The methods and compositions also may be used as a diagnostic to avoid surgical diagnostic procedures. The identification and use of such a panel of biomarker reagents provides a critical, more precise basis of knowledge to incorporate into pre-clinical and clinical diagnostic assays targeting these biomarkers.

Protein abundance levels of biomarkers in blood, in some embodiments, are dependent upon expression levels in tissues of origin (e.g., ovarian tumors), as well as rate of shedding into the blood and rate of clearance from the blood. While increased expression in a tumor often will correlate with increased abundance levels being observed in the blood, this is not necessarily always true. Therefore, the methods and compositions in one aspect refer to compositions that detect protein biomarkers and to protein assay methods. However, one of skill in the art, given the teachings contained herein, would readily understand that nucleic acid expression levels of the biomarkers and reagents and methods for their detections may be similarly practiced, without undue experimentation.

In one embodiment, the compositions and methods allow the detection and measurement of the protein levels or ratios of one or more “target” biomarkers of Table 1 in a biological sample, preferably a biological fluid. Diagnostic reagents that can detect and measure these target biomarkers and methods for evaluating the level or ratios of these target biomarkers vs. their level(s) in a variety of reference standards or controls of different conditions or stages in ovarian cancer are valuable tools in the early detection and monitoring of ovarian cancer.

### I. DEFINITIONS

“Patient” or “subject” as used herein means a female mammalian animal, including a human, a veterinary or farm animal, a domestic animal or pet, and animals normally used for clinical research. In one embodiment, the subject of these methods and compositions is a human.

By “biomarker” or “biomarker signature” as used herein is meant a single protein or a combination of proteins or peptide fragments thereof, the protein levels or relative protein levels or ratios of which significantly change (either in an increased or decreased manner) from the level or relative levels present in a subject having one physical condition or disease or disease stage from that of a reference standard representative of another physical condition or disease stage. Throughout this specification, wherever a particular biomarker is identified by name, it should be understood that the term “biomarker” includes those listed in Table 1 below, including any physiological molecular forms, or modified physiological molecular forms, isoforms, pro-forms, and peptide fragments thereof, unless otherwise specified. It is understood that all molecular forms useful in this context are physiological, e.g., naturally occurring in the species. Preferably the peptide fragments obtained from the biomarkers are unique sequences, such as those exemplified in Table 3. However, it is understood that fragments other than those explicitly identified may be obtained readily by one of skill in the art in view of the teachings provided herein.

TABLE 1
Selected Biomarkers of Ovarian Cancer

| Biomarker Name | Abbreviation |
|---|---|
| cathepsin D - 30 kDa | CTSD-30 kDa |
| cathepsin D - 52 kDa | CTSD-52 kDa |
| peroxieredoxin-6 | PRDX6 |
| chloride intracellular channel protein 1 | CLIC1 |
| Tropomyosin 1 | TPM1 |
| bisphosphoglycerate mutase | BPGM |
| proteasome subunit alpha type-7 | PSMA7 |
| aldose reductase | AKR1B1 |
| homeobox protein | HMX1 |
| melastatin 1 | TRPM1 |
| protein CutA | CUTA |
| SERPINB12 protein | SERPINB12 |

In one embodiment, at least one biomarker of Table 1 forms a suitable biomarker signature for use in the methods and compositions. In one embodiment, at least two biomarkers form a suitable biomarker signature for use in the methods and compositions. In another embodiment, at least three biomarkers form a suitable biomarker signature for use in the methods and compositions. In another embodiment, at least four biomarkers form a suitable biomarker signature for use in the methods and compositions. In another embodiment, at least five biomarkers form a suitable biomarker signature for use in the methods and compositions. In another embodiment, at least six biomarkers form a suitable biomarker signature for use in the methods and compositions. In another embodiment, at least seven biomarkers form a suitable biomarker signature for use in the methods and compositions. In another embodiment, at least eight biomarkers form a suitable biomarker signature for use in the methods and compositions. In still further embodiments, at least 9, at least 10, at least 11 or 12 of the biomarkers of Table 1 can be used alone or with additional biomarkers. Thus, from 1 to 12 of the biomarkers of Table 1, or ligands or reagents the interact with the biomarkers, can used in diagnostic panels or arrays or kits. In another embodiment, from 1 to 12 of the biomarker/ligands of Table 1 can be used with other known biomarkers for ovarian cancer and their ligands or reagents, so that the biomarker panel or array (or diagnostic reagent or kit) contains at least 20, 30, 40, 50 or at least 60 total biomarkers (or ligands) including any numbers there between. Specific biomarker signatures can include any combination of ovarian cancer biomarkers employing at least one biomarker (or its ligand) from Table 1 and including up to all 12 biomarkers in Table 1, in any combination with another biomarker, such as CA125. Still other biomarkers such as identified in the references cited herein may also be present in panels with the biomarkers of Table 1.

While these biomarkers may be used individually or in various combinations as signatures, it is contemplated that multiple molecular forms of each biomarker of Table 1 are similarly useful in the compositions and methods described herein. For example, the 30 kDa form of Cathepsin D (CTSD-30 kDa) is the heavy chain of the mature form of the enzyme. An alternative molecular form is the major form of Cathepsin D found in serum, i.e., the 52 kDa procathepsin D, consisting of a pro sequence+heavy chain+light chain. Still other modified molecular forms of CTSD and other biomarkers include protein modifications such as different glycosylation patterns and other conventional protein modifications of the biomarkers that occur in nature. In one embodiment, multiple molecular forms of the same protein biomarker parallel each other in patient samples. In another embodiment, one molecular form of a biomarker is a better biomarker for a condition than the other. In another embodiment, a ratio of the multiple molecular forms of the same biomarker forms a useful biomarker. Multiple molecular forms of these biomarkers can occur in blood or other biological samples. In certain embodiments, the presence of one or multiple specific molecular forms of the same biomarker, or a ratio of same, rather than overall protein levels is useful as a biomarker for a particular condition specified herein. One skilled in the art may readily reproduce the compositions and methods described herein by use of the sequences of the biomarkers, all of which are publicly available from conventional sources, such as GENBANK, UniProt or NCBI.

By “isoform” or “multiple molecular form” is meant an alternative expression product or variant of a single gene in a given species, including forms generated by alternative splicing, single nucleotide polymorphisms, alternative promoter usage, alternative translation initiation and small genetic differences between alleles of the same gene. See, e.g., human tropomyosin 1, also known as tropomyosin alpha-1 (NCBI reference P09493.2), which has isoforms 1-7 (see NCBI reference Nos. NP_{—}001018005.1, NP_{—}001018007.1, NP_{—}001018004.1, NP_{—}001018006.1, NP_{—}000357.3, NP_{—}001018008.1 and ACL14506.1, respectively) among others. See also UniProt Ref Q1ZYL5 and UniProt Ref B7Z596.

By “homologous protein” is meant an alternative form of a related protein produced from a related gene, or a protein produced from a different gene having a percent sequence similarity or identity of greater than 20%.

“Reference standard” as used herein refers to the source of the reference biomarker levels. The “reference standard” is preferably provided by using the same assay technique as is used for measurement of the subject's biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a protein abundance profile or protein level profile derived from the same biomarker or biomarkers in a reference subject or reference population. In an embodiment, in which expression of nucleic acid sequences encoding the biomarkers is desired to be evaluated, the reference standard can be an expression level of one or more biomarkers or an expression profile.

“Reference subject” or “Reference Population” defines the source of the reference standard. In one embodiment, the reference is a human subject or a population of subjects having no ovarian cancer, i.e., healthy controls or negative controls. In yet another embodiment, the reference is a human subject or population of subjects with one or more clinical indicators of ovarian cancer, but who did not develop ovarian cancer. In still another embodiment, the reference is a human subject or a population of subjects having benign ovarian nodules or cysts. In still another embodiment, the reference is a human subject or a population of subjects who had ovarian cancer, following surgical removal of an ovarian tumor. In another embodiment, the reference is a human subject or a population of subjects who had ovarian cancer and were evaluated for biomarker levels prior to surgical removal of an ovarian tumor. Similarly, in another embodiment, the reference is a human subject or a population of subjects evaluated for biomarker levels following therapeutic treatment for ovarian cancer. In still another embodiment, the reference is a human subject or a population of subjects prior to therapeutic treatment for an ovarian cancer. Similarly, in another embodiment, the reference human subject or a population of subjects without ovarian cancer but which tests positive for a protein level of CA-125. Similarly, in another embodiment, the reference human subject or a population of subjects with ovarian cancer but which tests negative for a protein level of CA125. In still other embodiments of methods described herein, the reference is obtained from the same test subject who provided a temporally earlier biological sample. That sample can be pre- or post-therapy or pre- or post-surgery.

Other potential reference standards are obtained from a reference that is a human subject or a population of subjects having early stage ovarian cancer. In another embodiment the reference is a human subject or a population of subjects having advanced stage ovarian cancer. In still another embodiment, the reference is a human subject or a population of subjects having a subtype of epithelial ovarian cancer. In still another embodiment, the reference is a human subject or a population of subjects having serous ovarian cancer or serous papillary adenocarcinoma. In still another embodiment, the reference is a human subject or a population of subjects having mucinous ovarian cancer. In still another embodiment, the reference is a human subject or a population of subjects having clear cell ovarian cancer. In still another embodiment, the reference is a subject or a population of subjects having endometrioid ovarian cancer. In another embodiment, the reference is a human subject or a population of subjects having Mullerian ovarian cancer. In another embodiment, the reference is a human subject or a population of subjects having undifferentiated ovarian cancer or an ovarian sarcoma. In another embodiment, the reference standard is a combination of two or more of the above reference standards.

Selection of the particular class of reference standards, reference population, biomarker levels or profiles depends upon the use to which the diagnostic/monitoring methods and compositions are to be put by the physician and the desired result, e.g., initial diagnosis of ovarian cancer or other ovarian condition, clinical management of patients with ovarian cancer after initial diagnosis, including, but not limited to, monitoring for reoccurrence of disease or monitoring remission or progression of the cancer and either before, during or after therapeutic or surgical intervention, selecting among therapeutic protocols for individual patients, monitoring for development of toxicity or other complications of therapy, predicting development of therapeutic resistance, and the like. Such reference standards or controls are the types that are commonly used in similar diagnostic assays for other biomarkers.

“Sample” as used herein means any biological fluid or tissue that contains the ovarian cancer biomarkers of Table 1. The most suitable samples for use in the methods and with the compositions are samples which require minimal invasion for testing, e.g., blood samples, including serum, plasma, whole blood, and circulating tumor cells. It is also anticipated that other biological fluids, such as saliva or urine, vaginal or cervical secretions, and ascites fluids or peritoneal fluid may be similarly evaluated by the methods described herein. Also, circulating tumor cells or fluids containing them are also suitable samples for evaluation in certain embodiments of this invention. The samples may include biopsy tissue, tumor tissue, surgical tissue, circulating tumor cells, or other tissue. Such samples may further be diluted with saline, buffer or a physiologically acceptable diluent. Alternatively, such samples are concentrated by conventional means. In certain embodiments, e.g., those in which expression levels of nucleic acid sequences encoding the biomarkers are desired to be evaluated, the samples may include biopsy tissue, surgical tissue, circulating tumor cells, or other tissue.

By “significant change in protein level” is meant an increased protein level of a selected biomarker in comparison to that of the selected reference standard or control or relative to a predetermined cutpoint; a decreased protein level of a selected biomarker in comparison to that of the selected reference or control or relative to a predetermined cutpoint; or a combination of a pattern or relative pattern of certain increased and/or decreased biomarkers. As shown in Table 5, in some embodiments, biomarkers are used to determine a cutpoint, which is a relative or absolute value for the level of a biomarker in a reference subject or reference population. The cut point is determined such that the false positives and false negatives are optimized. That is, if false negatives are less of a problem than false positives, the cut point can be adjusted accordingly.

The degree of change in biomarker protein level can vary with each individual and is subject to variation with each population. For example, in one embodiment, a large change, e.g., 2-3 fold increase or decrease in protein levels of a small number of biomarkers, e.g., from 1 to 9 characteristic biomarkers, is statistically significant. In another embodiment, a smaller relative change in 10 or more (i.e., about 10, 20, 24, 29, or 30 or more biomarkers) is statistically significant. The degree of change in any biomarker(s) expression varies with the condition, such as type of ovarian cancer and with the size or spread of the cancer or solid tumor. The degree of change also varies with the immune response of the individual and is subject to variation with each individual For example, in one embodiment of this invention, a change at or greater than a 1.2 fold increase or decrease in protein level of a biomarker or more than two such biomarkers, or even 3 or more biomarkers, is statistically significant. In another embodiment, a larger change, e.g., at or greater than a 1.5 fold, greater than 1.7 fold or greater than 2.0 fold increase or a decrease in expression of a biomarker(s) is statistically significant. This is particularly true for cancers without solid tumors. Still alternatively, if a single biomarker protein level is significantly increased in biological samples which normally do not contain measurable protein levels of the biomarker, such increase in a single biomarker level may alone be statistically significant. Conversely, if a single biomarker protein level is normally decreased or not significantly measurable in certain biological samples which normally do contain measurable protein levels of the biomarker, such decrease in protein level of a single biomarker may alone be statistically significant.

A change in protein level of a biomarker required for diagnosis or detection by the methods described herein refers to a biomarker whose protein level is increased or decreased in a subject having a condition or suffering from a disease, specifically ovarian cancer, relative to its expression in a reference subject or reference standard. Biomarkers may also be increased or decreased in protein level at different stages of the same disease or condition. The protein levels of specific biomarkers differ between normal subjects and subjects suffering from a disease, benign ovarian nodules, or cancer, or between various stages of the same disease. Protein levels of specific biomarkers differ between pre-surgery and post-surgery patients with ovarian cancer. Such differences in biomarker levels include both quantitative, as well as qualitative, differences in the temporal or relative protein level or abundance patterns among, for example, biological samples of normal and diseased subjects, or among biological samples which have undergone different disease events or disease stages. For the purpose of this invention, a significant change in biomarker protein levels when compared to a reference standard is considered to be present when there is a statistically significant (p<0.05) difference in biomarker protein level between the subject and reference standard or profile, or significantly different relative to a predetermined cut-point.

For example, in one embodiment, the test subject's biomarker(s) levels are compared with a healthy reference standard. If the subject has ovarian cancer, the biomarker(s) of Table 1 will typically show a change in protein level from the levels in the healthy reference standard, thus permitting diagnosis of ovarian cancer. In another example, the biomarker(s) of Table 1 differentially change in protein level (either by increased or decreased protein level) when the biomarker levels or relative levels from the sample of a subject having one of the following conditions is compared to a reference subject or population having another of the following physical conditions. These “conditions” include no ovarian cancer, the presence of benign ovarian nodules, the presence of an ovarian cancer or subtype, the condition following surgical removal of an ovarian tumor; the condition prior to surgical removal of an ovarian tumor; the condition following a specific therapeutic treatment for an ovarian tumor; the condition prior to a specific therapeutic treatment for an ovarian tumor. It is further anticipated that the biomarker(s) expression levels may change and the changes may be detected during treatment for ovarian cancer. In another embodiment, a condition includes that of a subject having undiagnosed clinical symptoms of abdominal pain or other abdominal condition of unknown origin. Still other embodiments of “conditions” as defined above include early stage ovarian cancer; advanced stage ovarian cancer, a subtype of epithelial ovarian cancer, serous ovarian cancer; mucinous ovarian cancer, clear cell ovarian cancer, endometrioid ovarian cancer, Mullerian ovarian cancer; undifferentiated ovarian cancer, serous papillary adenocarcinoma; and sarcoma.

The term “ligand” refers with regard to protein biomarkers to a molecule that binds or complexes, with a biomarker protein, molecular form or peptide, such as an antibody, antibody mimic or equivalent that binds to or complexes with a biomarker of Table 1, a molecular form or fragment thereof. In certain embodiments, in which the biomarker expression is to be evaluated, the ligand can be a nucleotide sequence, e.g., polynucleotide or oligonucleotide, primer or probe.

As used herein, the term “antibody” refers to an intact immunoglobulin having two light and two heavy chains or fragments thereof capable of binding to a biomarker protein or a fragment of a biomarker protein. Thus a single isolated antibody or fragment may be a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, an Fv construct, a Fab construct, an Fc construct, a light chain variable or complementarity determining region (CDR) sequence, etc.

As used herein, “labels” or “reporter molecules” are chemical or biochemical moieties useful for labeling a ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to a ligand.

As used herein the term “cancer” refers to or describes the physiological condition in mammals that is typically characterized by unregulated cell growth. More specifically, as used herein, the term “cancer” means any ovarian cancer. In one embodiment, the ovarian cancer is an epithelial ovarian cancer or subtype as referred to in “conditions” above. In still an alternative embodiment, the cancer is an “early stage” (I or II) ovarian cancer. In still another embodiment, the cancer is a “late stage” (III or IV) ovarian cancer.

The term “tumor,” as used herein, refers to all neoplastic cell growth and proliferation, whether malignant or benign, and all pre-cancerous and cancerous cells and tissues.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

The term “microarray” refers to an ordered arrangement of binding/complexing array elements or ligands, e.g. antibodies, on a substrate.

In the context of the compositions and methods described herein, reference to “at least two,” “at least five,” etc. of the biomarkers listed in any particular biomarker set means any and all combinations of the biomarkers identified. Specific biomarkers for the biomarker profile do not have to be in rank order in Table 1 and may be any biomarker, fragment or molecular form, as discussed herein.

By “significant change in expression” is meant an upregulation in the expression level of a nucleic acid sequence, e.g., genes or transcript, encoding a selected biomarker, in comparison to the selected reference standard or control; a downregulation in the expression level of a nucleic acid sequence, e.g., genes or transcript, encoding a selected biomarker, in comparison to the selected reference standard or control; or a combination of a pattern or relative pattern of certain upregulated and/or down regulated biomarker genes. The degree of change in biomarker expression can vary with each individual as stated above for protein biomarkers.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

One skilled in the art may readily reproduce the compositions and methods described herein by use of the amino acid sequences of the biomarkers and other molecular forms, which are publicly available from conventional sources.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### II. BIOMARKERS AND BIOMARKER SIGNATURES USEFUL IN THE METHODS AND COMPOSITIONS

The “targets” of the compositions and methods of these inventions include, in one aspect, biomarkers listed in Table 1, fragments, particularly unique fragments thereof, and molecular forms thereof. In certain embodiments, superior diagnostic tests for diagnosing the existence of ovarian cancer utilize at least one of the ligands that bind or complex with one of biomarkers of Table 1, or one of the fragments or molecular forms thereof. In other embodiments, superior diagnostic tests for distinguishing ovarian cancer from one of the conditions recited above utilize multiple ligands, each individually detecting a different specific target biomarker identified herein, or isoform, modified form or peptide thereof. In still other methods, no ligand is necessary, e.g., MRM assays.

In one embodiment the target biomarker of the methods and compositions described herein is cathepsin D, preferably the mature 30 kDa molecular form of the biomarker (CTSD-30 kDa). In another embodiment the target biomarker of the methods and compositions described herein is cathepsin D, preferably 52 kDa molecular form of the biomarker (CTSD-52 kDa). The amino acid sequences for CTSD and its molecular forms are publically available, such as in GENBANK. Certain fragments of CTSD-30 kDa or CTSD-52 kDa that may be useful as targets in the methods and compositions described herein include one or more peptide fragments, such as, but not limited to, those identified in Table 3. It should be understood that, depending upon the context, any reference to CTSD herein also refers to a peptide and the molecular form thereof, as well as the nucleotide sequences encoding CTSD and/or any of the peptides or forms.

In one embodiment the target biomarker of the methods and compositions described herein is chloride intracellular channel protein 1 (CLIC1). The amino acid sequence for CLIC1 is publically available, such as in GENBANK. Certain fragments of CLIC1 that may be useful as targets in the methods and compositions described herein include one or more peptide fragments, such as, but not limited to, those identified in Table 3. It should be understood that, depending upon the context, any reference to CLIC1 herein also refers to any of these peptides, or any molecular form of the biomarker, as well as the nucleotide sequences encoding CLIC1 and/or any of the peptides.

In one embodiment the target biomarker of the methods and compositions described herein is peroxieredoxin-6 (PRDX6). The amino acid sequence for PRDX6 is publically available, such as in GENBANK. Certain fragments of PRDX6 that may be useful as targets in the methods and compositions described herein include one or more peptide fragments, such as, but not limited to, those identified in Table 3. It should be understood that, depending upon the context, any reference to PRDX6 herein also refers to any of these peptides, any molecular forms, as well as the nucleotide sequences encoding PRDX6 and/or any of the peptides.

In one embodiment the target biomarker of the methods and compositions described herein is Tropomyosin 1 (TPM1), including its isoforms, such as TPM1, isoform 6. The amino acid sequence for TPM1 is publically available, such as in GENBANK. Certain fragments of TPM1 may also be useful as targets in the methods and compositions described herein. It should be understood that, depending upon the context, any reference to PRDX6 herein also refers to any of its peptides or molecular forms, as well as the nucleotide sequences encoding PRDX6 and/or any of the peptides.

In one embodiment the target biomarker of the methods and compositions described herein is bisphosphoglycerate mutase (BPGM). The amino acid sequence for BPGM is publically available, such as in GENBANK. Certain fragments of BPGM may also be useful as targets in the methods and compositions described herein. It should be understood that, depending upon the context, any reference to BPGM herein also refers to any of its peptides, any molecular forms, as well as the nucleotide sequences encoding BPGM and/or any of the peptides.

In one embodiment the target biomarker of the methods and compositions described herein is proteasome subunit alpha type-7 (PSMA7). The amino acid sequence for PSMA7 is publically available, such as in GENBANK. Certain fragments of PSMA7 may also be useful as targets in the methods and compositions described herein. It should be understood that, depending upon the context, any reference to PSMA7 herein also refers to any of its peptides, any molecular forms, as well as the nucleotide sequences encoding PSMA7 and/or any of the peptides.

In one embodiment the target biomarker of the methods and compositions described herein is aldose reductase (AKR1B1). The amino acid sequence for AKR1B1 is publically available, such as in GENBANK. Certain fragments of AKR1B1 that may be useful as targets in the methods and compositions described herein include one or more peptide fragments, such as, but not limited to, those identified in Table 3. It should be understood that, depending upon the context, any reference to AKR1B1 herein also refers to any of these peptides, any molecular forms, as well as the nucleotide sequences encoding AKR1B1 and/or any of the peptides.

In one embodiment the target biomarker of the methods and compositions described herein is homeobox protein (HMX1). The amino acid sequence for HMX1 is publically available, such as in GENBANK. Certain fragments of HMX1 that may be useful as targets in the methods and compositions described herein include one or more peptide fragments, such as, but not limited to, those identified in Table 3. It should be understood that, depending upon the context, any reference to HMX1 herein also refers to any of these peptides, any molecular forms, as well as the nucleotide sequences encoding HMX1 and/or any of the peptides.

In one embodiment the target biomarker of the methods and compositions described herein is melastatin 1 (TRPM1). The amino acid sequence for TRPM1 is publically available, such as in GENBANK. Certain fragments of TRPM1 that may be useful as targets in the methods and compositions described herein include one or more peptide fragments, such as, but not limited to, those identified in Table 3. It should be understood that, depending upon the context, any reference to TRPM1 herein also refers to any of these peptides, any molecular forms, as well as the nucleotide sequences encoding TRPM1 and/or any of the peptides.

In one embodiment the target biomarker of the methods and compositions described herein is protein CutA (CUTA). The amino acid sequence for CUTA is publically available, such as in GENBANK. Certain fragments of CUTA that may be useful as targets in the methods and compositions described herein one or more peptide fragments, such as, but not limited to, those identified in Table 3. It should be understood that, depending upon the context, any reference to CUTA herein also refers to any of these peptides, any molecular fauns, as well as the nucleotide sequences encoding CUTA and/or any of the peptides.

In one embodiment the target biomarker of the methods and compositions described herein is SERPINB12 protein (SERPINB12). The amino acid sequence for SERPINB12 is publically available, such as in GENBANK. Certain fragments of SERPINB 12 that may be useful as targets in the methods and compositions described herein include one or more peptide fragments, such as, but not limited to, those identified in Table 3. It should be understood that, depending upon the context, any reference to SERPINB12 herein also refers to any of the peptides, any molecular forms, as well as the nucleotide sequences encoding SERPINB12 and/or any of the peptides.

In still another embodiment, the biomarkers targeted by the methods and compositions described herein includes various combinations of these target biomarkers and/or fragments thereof.

In another embodiment, a combination of ligands is used to identify biomarkers including the known ovarian cancer biomarker, CA125 or any of its molecular forms, in combination with one or more of the above-noted biomarkers of Table 1. In one embodiment, the methods and compositions employ ligands that target multiple biomarkers. The multiple biomarker combinations include, without limitation, or consist of, the following exemplary combinations of biomarkers or combinations that include different molecular forms of the same biomarker, for diagnosis of ovarian cancer or for monitoring the progression of the severity of disease or remission of disease:

CTSD (CTSD-30 kDa or CTSD-52 kDa) with one or more of CLIC1, PRDX6, TPM1, BPGM, PSMA7, AKRIB1, HMX1, TRPM1, CUTA, SERPINB12; or

CTSD (CTSD-30 kDa or CTSD-52 kDa) with one or more of CLIC1, PRDX6, TPM1, BPGM, PSMA7, AKRIB1, HMX1, TRPM1, CUTA, SERPINB12 and CA125; or

CTSD (CTSD-30 kDa or CTSD-52 kDa) with one or more of CLIC1, PRDX6, TPM1, BPGM, PSMA7, AKRIB1, HMX1, TRPM1, CUTA, SERPINB12 and an additional known ovarian cancer biomarker;

CLIC1 with one or more of CTSD (CTSD-30 kDa or CTSD-52 kDa), PRDX6, TPM1, BPGM, PSMA7, AKRIB1, HMX1, TRPM1, CUTA, SERPINB12;

CLIC1 with one or more of CTSD (CTSD-30 kDa or CTSD-52 kDa), PRDX6, TPM1, BPGM, PSMA7, AKRIB1, HMX1, TRPM1, CUTA, SERPINB12 and CA125; or

CLIC1 with one or more of CTSD (CTSD-30 kDa or CTSD-52 kDa), PRDX6, TPM1, BPGM, PSMA7, AKRIB1, HMX1, TRPM1, CUTA, SERPINB12 and an additional known ovarian cancer biomarker;

PRDX6 with one or more of CTSD (CTSD-30 kDa or CTSD-52 kDa), CLIC1, TPM1, BPGM, PSMA7, AKRIB1, HMX1, TRPM1, CUTA, SERPINB12;

PRDX6 with one or more of CTSD (CTSD-30 kDa or CTSD-52 kDa), CLIC1, TPM1, BPGM, PSMA7, AKRIB1, HMX1, TRPM1, CUTA, SERPINB12 and CA125; or

PRDX6 with one or more of CTSD (CTSD-30 kDa or CTSD-52 kDa), CLIC1, TPM1, BPGM, PSMA7, AKRIB1, HMX1, TRPM1, CUTA, SERPINB12 and an additional known ovarian cancer biomarker;

TPM1 with one or more of CTSD (CTSD-30 kDa or CTSD-52 kDa), CLIC1, PRDX6, BPGM, PSMA7, AKRIB1, HMX1, TRPM1, CUTA, SERPINB12;

TPM1 with one or more of CTSD (CTSD-30 kDa or CTSD-52 kDa), CLIC1, PRDX6, BPGM, PSMA7, AKRJB1, HMX1, TRPM1, CUTA, SERPINB12 and CA125; or

TPM1 with one or more of CTSD (CTSD-30 kDa or CTSD-52 kDa), CLIC1, PRDX6, BPGM, PSMA7, AKRIB1, HMX1, TRPM1, CUTA, SERPINB12 and an additional known ovarian cancer biomarker;

BPGM with one or more of CTSD (CTSD-30 kDa or CTSD-52 kDa), CLIC1, TPM1, PRDX6, PSMA7, AKRIB1, HMX1, TRPM1, CUTA, SERPINB12;

BPGM with one or more of CTSD (CTSD-30 kDa or CTSD-52 kDa), CLIC1, TPM1, PRDX6, PSMA7, AKRIB1, HMX1, TRPM1, CUTA, SERPINB12 and CA125; or

BPGM with one or more of CTSD (CTSD-30 kDa or CTSD-52 kDa), CLIC1, TPM1, PRDX6, PSMA7, AKRIB1, HMX1, TRPM1, CUTA, SERPINB12 and an additional known ovarian cancer biomarker;

PSMA7 with one or more of CTSD (CTSD-30 kDa or CTSD-52 kDa), CLIC1, PRDX6, BPGM, TPM1, AKRJB1, HMX1, TRPM1, CUTA, SERPINB12;

PSMA7 with one or more of CTSD (CTSD-30 kDa or CTSD-52 kDa), CLIC1, PRDX6, BPGM, TPM1, AKRIB1, HMX1, TRPM1, CUTA, SERPINB12 and CA125; or

PSMA7 with one or more of CTSD (CTSD-30 kDa or CTSD-52 kDa), CLIC1, PRDX6, BPGM, TPM1, AKRIB1, HMX1, TRPM1, CUTA, SERPINB12 and an additional known ovarian cancer biomarker;

AKRJB1 with one or more of CTSD (CTSD-30 kDa or CTSD-52 kDa), CLIC1, PRDX6, BPGM, TPM1, PSMA7, HMX1, TRPM1, CUTA, SERPINB12;

AKRIB1 with one or more of CTSD (CTSD-30 kDa or CTSD-52 kDa), CLIC1, PRDX6, BPGM, TPM1, PSMA7, HMX1, TRPM1, CUTA, SERPINB12 and CA125; or

AKRIB1 with one or more of CTSD (CTSD-30 kDa or CTSD-52 kDa), CLIC1, PRDX6, BPGM, TPM1, PSMA7, HMX1, TRPM1, CUTA, SERPINB12 and an additional known ovarian cancer biomarker;

HMX1 with one or more of CTSD (CTSD-30 kDa or CTSD-52 kDa), CLIC1, PRDX6, BPGM, TPM1, PSMA7, AKRIB1, TRPM1, CUTA, SERPINB12;

HMX1 with one or more of CTSD (CTSD-30 kDa or CTSD-52 kDa), CLIC1, PRDX6, BPGM, TPM1, PSMA7, AKRIB1, TRPM1, CUTA, SERPINB12 and CA125; or

HMX1 with one or more of CTSD (CTSD-30 kDa or CTSD-52 kDa), CLIC1, PRDX6, BPGM, TPM1, PSMA7, AKRIB1, TRPM1, CUTA, SERPINB12 and an additional known ovarian cancer biomarker;

TRPM1 with one or more of CTSD (CTSD-30 kDa or CTSD-52 kDa), CLIC1, PRDX6, BPGM, TPM1, PSMA7, AKRIB1, HMX1, CUTA, SERPINB12;

TRPM1 with one or more of CTSD (CTSD-30 kDa or CTSD-52 kDa), CLIC1, PRDX6, BPGM, TPM1, PSMA7, AKRIB1, HMX1, CUTA, SERPINB12 and CA125; or

TRPM1 with one or more of CTSD (CTSD-30 kDa or CTSD-52 kDa), CLIC1, PRDX6, BPGM, TPM1, PSMA7, AKRIB1, HMX1, CUTA, SERPINB12 and an additional known ovarian cancer biomarker;

CUTA with one or more of CTSD (CTSD-30 kDa or CTSD-52 kDa), CLIC1, PRDX6, BPGM, TPM1, PSMA7, AKRIB1, HMX1, TRPM1, SERPINB12;

CUTA with one or more of CTSD (CTSD-30 kDa or CTSD-52 kDa), CLIC1, PRDX6, BPGM, TPM1, PSMA7, AKRIB1, HMX1, TRPM1, SERPINB12 and CA125; or

CUTA with one or more of CTSD (CTSD-30 kDa or CTSD-52 kDa), CLIC1, PRDX6, BPGM, TPM1, PSMA7, AKRIB1, HMX1, TRPM1, SERPINB12 and an additional known ovarian cancer biomarker;

SERPINB 12 with one or more of CTSD (CTSD-30 kDa or CTSD-52 kDa), CLIC1, PRDX6, BPGM, TPM1, PSMA7, AKRIB1, HMX1, TRPM1, CUTA;

SERPINB12 with one or more of CTSD (CTSD-30 kDa or CTSD-52 kDa), CLIC1, PRDX6, BPGM, TPM1, PSMA7, AKRIB1, HMX1, TRPM1, CUTA and CA125; or

SERPINB12 with one or more of CTSD (CTSD-30 kDa or CTSD-52 kDa), CLIC1, PRDX6, BPGM, TPM1, PSMA7, AKRIB1, HMX1, TRPM1, CUTA and an additional known ovarian cancer biomarker. Still other combinations of the Table 1 low abundance biomarkers can be targeted in combination with other known ovarian cancer biomarkers to produce a desired signature for one of the ovarian cancer-related conditions described above.

In still another aspect, a biomarker combination includes, without limitation, or consists of, the following exemplary combinations of biomarkers for diagnosis of ovarian cancer or for monitoring the progression of the severity of disease or remission of disease:

CTSD-30 kDa, and/or CLIC1, and/or PRDX6, or

CTSD-30 kDa, CLIC1, and PRDX6;

CTSD-30 kDa, CLIC1, PRDX6 and TPM1;

CTSD-30 kDa, CLIC1, PRDX6 and BPGM,

CTSD-30 kDa, CLIC1, PRDX6 and PSMA7,

CTSD-30 kDa, CLIC1, PRDX6, TPM1 and BPGM,

CTSD-30 kDa, CLIC1, PRDX6, TPM1 and PSMA7,

CTSD-30 kDa, CLIC1, PRDX6, TPM1, BPGM and PSMA7,

or an isoform, pro-form, modified molecular form, or peptide fragment thereof.

For example, among desirable biomarker signatures are signatures that comprise, or consist of, at least 3, 4, 5, 6, 7, 8, 9, 10 or 11 of the biomarkers of Table 1, including optionally CA125 or any other known ovarian cancer biomarker or molecular forms or peptides thereof. It is contemplated that even the high abundance biomarkers indicated in Table 2 may be useful when combined in a panel or signature with the low abundance biomarker(s) of Table 1.

TABLE 2
Selected human proteins shed by ovarian TOV-112D tumors into SCID mouse blood

| | | | | | | |
|---|---|---|---|---|---|---|
| Uniprot | | | | Peptides | MRM | |
| ID | Name | Descriptive Name | Hu/In^{a} | Verified^{b} | Assay | Comments |

Medium and High Abundance Serum Proteins

| | | | | | | |
|---|---|---|---|---|---|---|
| Q14624 | ITIH4 | Inter-alpha-trypsin | 5/5 | n.d. | n.d. | Biomarker for |
| | | inhibitor heavy | | | | ovarian cancer¹⁴ |
| | | chain H4 | | | | |
| P02647 | APOA1 | Apolipoprotein A-I | 10/0 | n.d. | n.d. | Biomarker for |
| | | | | | | ovarian cancer^{14, 64} |
| P61769 | B2M | Beta-2- | 1/0 | n.d. | n.d. | Overexpressed in |
| | | microglobulin | | | | many cancer |
| | | | | | | cells, including |
| | | | | | | ovarian⁶⁵ |
| P02766 | TTR | Transthyretin | 4/0 | n.d. | n.d. | Biomarker for |
| | | | | | | ovarian cancer^{14, 64} |
| P02787 | TF | Transferrin | 4/1 | n.d. | n.d. | Biomarker for |
| | | | | | | ovarian cancer⁶⁴ |
| P07195 | LDHB | L-lactate | 1/6 | n.d. | n.d. | Increased in |
| | | dehydrogenase B | | | | tissue cytosol |
| | | chain | | | | from patients |
| | | | | | | with ovarian |
| | | | | | | cancer⁶⁶ |
| P23528 | CFL1 | Cofilin-1 | 1/7 | n.d. | n.d. | Identified in |
| | | | | | | TOV-112D |
| | | | | | | secretome³⁵ |
| P05090 | APOD | Apolipoprotein D | 3/0 | n.d. | n.d. | Increased in |
| | | | | | | ovarian cancer |
| | | | | | | tumors⁶⁷ |
| P10909 | CLU | Clusterin | 7/0 | 8 | yes | Higher level in |
| | | | | | | serum of ovarian |
| | | | | | | cancer patients³⁵ |

Candidate Biomarkers Evaluated

| | | | | | | |
|---|---|---|---|---|---|---|
| P07339 | CTSD | Cathepsin D | 3/1 | 8 | yes | Prognostic factor |
| | | | | | | for ovarian |
| | | | | | | cancer⁵¹ |
| O00299 | CLIC1 | Chloride | 1/4 | 7 | yes | Identified in |
| | | intracellular | | | | TOV-112D |
| | | channel protein 1 | | | | secretome² |
| P15121 | AKR1B1 | Aldose reductase | 1/3 | 1 | no | Increase in breast, |
| | | | | | | ovarian, cervical, |
| | | | | | | and rectal cancer |
| | | | | | | tissues⁵³ |
| Q9NP08 | HMX1 | Homeobox protein | 1/0 | 1 | no | |
| | | HMX1 | | | | |
| Q7Z4N2 | TRPM1 | Melastatin 1 | 1/0 | 0 | no | |
| O60888 | CUTA | Protein CutA | 1/0 | 0 | no | Identified in |
| | | | | | | TOV-112D |
| | | | | | | secretome³⁵ |
| Q3SYB4 | SERPINB12 | SERPINB12 | 1/0 | 0 | no | |
| | | protein | | | | |
| P30041 | PRDX6 | Peroxiredoxin-6 | 0/4 | 6 | yes | Identified in |
| | | | | | | TOV-112D |
| | | | | | | secretome³⁵ |

^{a}Number of unique human/indistinguishable peptides. MS/MS spectra of proteins identified by a single human peptide are shown in Supplemental FIG. 1 of Tang et al, 2012.
^{b}Number of peptides successfully verified in targeted Orbigrap MS/MS analysis of pooled advanced ovarian cancer sera.
n.d., not determined

Table 3A through 3C form a single tabular report showing Sequest peptide identification of selected human proteins shed by ovarian TOV-112D tumors into SCID mouse.

TABLE 3A

| | Uniprot | | | Sequence | Spectrum | Seq. |
|---|---|---|---|---|---|---|
| # | ID | Name | Description | Count | Count | Coverage |
| 1 | Q14624 | ITIH4 | Inter-alpha trypsin | 10 | 2953 | 13.2 |
| | | | inhibitor heavy chain | | | |
| | | | H4 | | | |
| 2 | P02647 | APOA1 | Apolipoprotein A-1 | 10 | 3014 | 43.4 |
| 3 | P61769 | B2M | Beta-2-microglobulin | 1 | 9 | 8.4 |
| 4 | P02766 | TTR | Transthyretin | 4 | 19 | 47.6 |
| 5 | P02787 | TF | Transferrin | 5 | 8 | 9.3 |
| 6 | P07195 | LDHB | L-lactate | 7 | 256 | 23.7 |
| | | | dehydrogenase B | | | |
| | | | chain | | | |
| 7 | P23528 | CFL1 | Cofilin-1 | 8 | 70 | 51.5 |
| 8 | P10909 | CLU | Clusterin | 7 | 34 | 16.7 |
| 9 | P05090 | APOD | Apolipoprotein D | 3 | 9 | 20.1 |
| 10 | P07339 | CTSD | Cathepsin D | 4 | 75 | 15.5 |
| 11 | O00299 | CLIC1 | Chloride intracellular | 5 | 5 | 27.9 |
| | | | channel protein 1 | | | |
| 12 | P15121 | AKR1B1 | Aldose reductase | 4 | 13 | 21 |
| 13 | Q9NP08 | HMX1 | Homeobox protein | 1 | 1 | 4.6 |
| | | | HMX1 | | | |
| 14 | Q7Z4N2 | TRPM1 | Melastatin 1 | 1 | 1 | 1 |
| 15 | O60888 | CUTA | Protein CutA | 1 | 1 | 15.1 |
| 16 | Q3SYB4 | SERPINB12 | SERPINB12 protein | 1 | 1 | 2.4 |

TABLE 3B

| # | MolWt | pI | ObsM+H+ | ppm | z | XCorr | DeltCN |
|---|---|---|---|---|---|---|---|
| 1 | 103358 | 7 | 877.444 | −0.9 | 2 | 2.5315 | 0.0188 |
| | | | 1452.7358 | 2 | 2 | 5.0242 | 0.3996 |
| | | | 933.543 | 2.8 | 2 | 1.8485 | 0.0218 |
| | | | 1811.959 | 5.1 | 2 | 4.8543 | 0.2937 |
| | | | 1256.591 | 2.3 | 2 | 2.4519 | 0.2073 |
| | | | 1722.839 | 1.3 | 2 | 4.4757 | 0.3792 |
| | | | 1017.5475 | −0.1 | 2 | 3.0339 | 0.3329 |
| | | | 1204.6698 | 1.1 | 3 | 3.3685 | 0.1134 |
| | | | 1144.623 | 1.9 | 2 | 3.7916 | 0.414 |
| | | | 2415.1867 | 3.3 | 3 | 3.4237 | 0.2848 |
| 2 | 30778 | 5.8 | 1215.624 | 2 | 2 | 3.16 | 0.3214 |
| | | | 1235.69265 | 3.6 | 2 | 3.7731 | 0.3692 |
| | | | 1612.789 | 2.3 | 2 | 4.5011 | 0.4187 |
| | | | 813.447 | 0.7 | 2 | 2.1938 | 0.0486 |
| | | | 1252.62865 | 6.3 | 2 | 2.3686 | 0.2111 |
| | | | 1386.7208 | 4.1 | 2 | 4.8366 | 0.4728 |
| | | | 1400.674 | 3.4 | 2 | 4.0743 | 0.4676 |
| | | | 1932.939 | 2.7 | 2 | 4.4915 | 0.4571 |
| | | | 1230.7147 | 4.5 | 2 | 2.9204 | 0.203 |
| | | | 1301.651 | 2 | 2 | 3.1564 | 0.456 |
| 3 | 13715 | 6.5 | 1122.6293 | 2.4 | 2 | 2.8905 | 0.1352 |
| 4 | 15887 | 5.7 | 1394.629 | 4.8 | 2 | 4.1445 | 0.4115 |
| | | | 2455.15565 | 1.9 | 3 | 6.6073 | 0.5253 |
| | | | 1366.764 | 3.7 | 2 | 3.8033 | 0.3815 |
| | | | 2360.24865 | 4.4 | 2 | 5.1587 | 0.4571 |
| 5 | 77050 | 7.1 | 2070.03365 | 2.2 | 2 | 5.0676 | 0.2878 |
| | | | 1629.81865 | 1.7 | 2 | 3.2168 | 0.3047 |
| | | | 1273.648 | −4.3 | 2 | 3.2423 | 0.365 |
| | | | 1249.607 | 0.9 | 2 | 2.6172 | 0.191 |
| | | | 1581.757 | −0.5 | 2 | 3.6935 | 0.2871 |
| 6 | 36507 | 6.1 | 957.6145 | 1.4 | 2 | 2.0985 | 0.005 |
| | | | 913.58453 | 1.8 | 2 | 2.8624 | 0.2956 |
| | | | 1694.89365 | −1.3 | 2 | 3.7462 | 0.2966 |
| | | | 1176.597 | 6.4 | 2 | 2.8992 | 0.1587 |
| | | | 1629.869 | 6.7 | 2 | 5.0858 | 0.4016 |
| | | | 959.558 | 6.2 | 2 | 2.9135 | 0.1164 |
| | | | 1290.652 | 3.8 | 2 | 3.5928 | 0.4448 |
| 7 | 18371 | 8.1 | 1223.6381 | 3.3 | 2 | 3.2213 | 0.3483 |
| | | | 1351.737 | 5.9 | 2 | 2.5279 | 0.0574 |
| | | | 2166.11165 | 7 | 2 | 6.2231 | 0.5645 |
| | | | 1561.73 | −1 | 2 | 4.0859 | 0.0867 |
| | | | 1832.8549 | −2.5 | 3 | 4.0433 | 0.2656 |
| | | | 1990.0759 | 3.8 | 3 | 4.2352 | 0.3407 |
| | | | 1130.647 | 4.8 | 2 | 3.9177 | 0.3099 |
| 8 | 52495 | 6.3 | 1873.99665 | 3.3 | 2 | 5.3043 | 0.4852 |
| | | | 1117.618 | 7.2 | 2 | 2.7757 | 0.1959 |
| | | | 1245.713 | 6.5 | 2 | 3.1696 | 0.2532 |
| | | | 1393.7 | 3 | 2 | 4.0304 | 0.37 |
| | | | 1288.647 | 7.1 | 2 | 3.2366 | 0.2778 |
| | | | 1245.70965 | 3.8 | 2 | 2.8017 | 0.1448 |
| | | | 2314.18165 | 1.9 | 3 | 2.8497 | 0.116 |
| 9 | 21276 | 5.1 | 1699.822 | 6.6 | 2 | 3.8704 | 0.3679 |
| | | | 1230.67 | 1 | 2 | 3.4477 | 0.2192 |
| | | | 1423.7522 | 6.7 | 2 | 2.9596 | 0.2164 |
| 10 | 44552 | 6.5 | 2047.05365 | −3.2 | 2 | 3.767 | 0.352 |
| | | | 1239.62155 | 2.1 | 2 | 3.6871 | 0.3827 |
| | | | 1803.81865 | 6.7 | 2 | 4.647 | 0.073 |
| | | | 1959.043 | 3.2 | 2 | 4.5561 | 0.4371 |
| 11 | 26792 | 5.2 | 1975.012 | −4.3 | 2 | 5.105 | 0.0375 |
| | | | 1281.667 | −1.2 | 2 | 3.6909 | 0.3248 |
| | | | 1844.98 | 2.6 | 2 | 4.3598 | 0.5206 |
| | | | 1328.6461 | 1.5 | 2 | 2.811 | 0.2444 |
| | | | 1065.617 | 0.5 | 2 | 2.4933 | 0.0695 |
| 12 | 35722 | 7 | 3262.67605 | 0.6 | 3 | 3.2635 | 0.3584 |
| | | | 1131.624 | 0.8 | 2 | 2.571 | 0.0728 |
| | | | 901.5472 | 0.7 | 2 | 2.6268 | 0.0717 |
| | | | 2260.14765 | 2 | 2 | 5.4665 | 0.4768 |
| 13 | 39225 | 11.6 | 1372.62865 | −4.8 | 2 | 3.0886 | 0.0265 |
| 14 | 174417 | 6.9 | 1880.9133 | 3.5 | 2 | 3.4248 | 0.0074 |
| 15 | 19116 | 5.5 | 3140.62965 | 3 | 3 | 6.4221 | 0.4185 |
| 16 | 48446 | 5.4 | 1153.58365 | −1 | 2 | 2.3854 | 0.213 |

TABLE 3C

| # | SpR | Sequence | SEQ ID NO |
|---|---|---|---|
| 1 | 12 | K.GSEMVVAGK.L | 20 |
| | 1 | K.NGIDIYSLTVDSR.V | 21 |
| | 2 | K.NVVFVIDK.S | 22 |
| | 1 | K.SPEQQETVLDGNLIIR.Y | 23 |
| | 1 | K.YIFHNFMER.L | 24 |
| | 1 | R.ANTVQEATFQM*ELPK.K | 25 |
| | 2 | R.FAHTVVTSR.V | 26 |
| | 2 | R.FKPTLSQQQK.S | 27 |
| | 1 | R.GPDVLTATVSGK.L | 28 |
| | 1 | R.QGPVNLLSDPEQGVEVTGQYER.E | 29 |
| 2 | 1 | K.ATEHLSTLSEK.A | 30 |
| | 1 | K.DLATVYVDVLK.D | 31 |
| | 1 | K.LLDNWDSVTSTFSK.L | 32 |
| | 40 | K.PALEDLR.Q | 33 |
| | 19 | K.VQPYLDDFQK.K | 34 |
| | 1 | K.VSFLSALEEYTK.K | 35 |
| | 1 | R.DYVSQFEGSALGK.Q | 36 |
| | 1 | R.EQLGPVTQEFWDNLEK.E | 37 |
| | 1 | R.QGLLPVLESFK.V | 38 |
| | 1 | R.THLAPYSDELR.Q | 39 |
| 3 | 1 | R.VNHVTLSQPK.I | 40 |
| 4 | 1 | K.AADDTWEPFASGK.T | 41 |
| | 1 | K.TSESELHGLTTEEEFVEGIYK.V | 42 |
| | 1 | R.GSPAINVAVHVFR.K | 43 |
| | 1 | R.YTIAALLSPYSYSTTAVVTNPK.E | 44 |
| 5 | 1 | K.EDLIWELLNQAQEHFGK.D | 45 |
| | 1 | K.EDPQTFYYAVAVVK.K | 46 |
| | 1 | K.HSTIFENLANK.A | 47 |
| | 6 | K.SASDLTWDNLK.G | 48 |
| | 1 | R.DQYELLCLDNTR.K | 49 |
| 6 | 21 | K.FIIPQWK.Y | 50 |
| | 1 | K.IVVVTAGVR.Q | 51 |
| | 1 | K.LIAPVAEEEATVPNNK.I | 52 |
| | 1 | K. SADTLWDIQK.D | 53 |
| | 1 | K.SLADELALVDVLEDK.L | 54 |
| | 4 | R.GLTSVINQK.L | 55 |
| | 1 | R.VIGSGCNLDSAR.F | 56 |
| 7 | 1 | K.AVLFCLSEDK.K | 57 |
| | 6 | K.AVLFCLSEDKK.N | 58 |
| | 1 | K.EILVGDVGQTVDDPYATFVK.M | 59 |
| | 1 | K.HELQANCYEEVK.D | 60 |
| | 1 | K.HELQANCYEEVKDR.C | 61 |
| | 1 | K.KEDLVFIFWAPESAPLK.S | 62 |
| | 1 | K.LGGSAVISLEGK.P | 63 |
| 8 | 1 | K.LFDSDPITVTVPVEVSR.K | 18 |
| | 1 | K.TLLSNLEEAK.K | 64 |
| | 1 | K.TLLSNLEEAKK.K | 65 |
| | 1 | R.ASSIIDELFQDR.F | 66 |
| | 2 | R.ELDESLQVAER.L | 67 |
| | 3 | R.KTLLSNLEEAK.K | 68 |
| | 15 | R.VTTVASHTSDSDVPSGVTEVVVK.L | 69 |
| 9 | 1 | K.CPNPPVQENFDVNK.Y | 70 |
| | 1 | K.NLTSNNIDVK.K | 71 |
| | 11 | R.NPNLPPETVDSLK.N | 72 |
| 10 | 1 | K.AIGAVPLIQGEYM*IPCEK.V | 73 |
| | 1 | K.FDGILGMAYPR.I | 16 |
| | 1 | R.DPDAQPGGELM*LGGTDSK.Y | 74 |
| | 1 | R.ISVNNVLPVFDNLMQQK.L | 75 |
| 11 | 1 | K.FLDGNELTLADCNLLPK.L | 76 |
| | 1 | K.GVTFNVTTVDTK.R | 77 |
| | 1 | K.LAALNPESNTAGLDIFAK.F | 78 |
| | 2 | K.NSNPALNDNLEK.G | 79 |
| | 1 | R.LFM*VLWLK.G | 80 |
| 12 | 1 | K.GIVVTAYSPLGSPDRPWAKPEDPSLLEDPR.I | 81 |
| | 5 | K.M*PILGLGTWK.S | 82 |
| | 9 | K.TTQVLIR.F | 83 |
| | 1 | K.YKPAVNQIECHPYLTQEK.L | 84 |
| 13 | 3 | R.SGAAGGSGAGGAWPGGR.H | 85 |
| 14 | 25 | K.AQSHQLFAIIM*ECM*K.K | 86 |
| 15 | 1 | R.SVHPYEVAEVIALPVEQGNFPYLQWVR.Q | 87 |
| 16 | 10 | K.TDYTLSIANR.L | 88 |

Tables 4A through 4C form a single tabular report showing normalized MRM peak area values for CLIC1, CTSD, and PRDX6 peptides and their averages in all samples analyzed.

TABLE 4A

| | | | | CLIC1 | CLIC1 |
|---|---|---|---|---|---|
| | Sample | Sample | | GVTFNVTTVDTK | NSNPALNDNLEK |
| # | Classification | Code | Type Model | (641.3/877.5) | (664.8/1013.5) |
| 1 | Stage 3 | T 455 | Cancer | 0.779 | 0.760 |
| 2 | Stage 4 | T 474 | Cancer | 1.839 | 1.638 |
| 3 | Stage 3 | T 475 | Cancer | 0.877 | 0.753 |
| 4 | Stage 3 | T 476 | Cancer | 1.420 | 1.195 |
| 5 | Stage 3 | T 478 | Cancer | 0.605 | 0.348 |
| 6 | Stage 4 | T 482 | Cancer | 2.567 | 2.206 |
| 7 | Stage 3 | T 536 | Cancer | 1.612 | 1.751 |
| 8 | Stage 3 | T 539 | Cancer | 0.998 | 0.960 |
| 9 | Stage 3 | T 541 | Cancer | 0.062 | 0.066 |
| 10 | Stage 3 | T 543 | Cancer | 0.430 | 0.210 |
| 11 | Stage 3 | T 553 | Cancer | 1.558 | 1.610 |
| 12 | Stage 3 | T 556 | Cancer | 1.762 | 3.158 |
| 13 | Stage 4 | T 557 | Cancer | 1.540 | 1.542 |
| 14 | Stage 3 | T 577 | Cancer | 0.288 | 0.394 |
| 15 | Stage 3 | T 600 | Cancer | 0.770 | 0.775 |
| 16 | Stage 3 | T 602 | Cancer | 0.215 | 0.234 |
| 17 | Stage 3 | T 603 | Cancer | 0.455 | 0.305 |
| 18 | Stage 3 | T 604 | Cancer | 0.192 | 0.094 |
| 19 | Benign | T B-23 | Normal | 0.234 | 0.263 |
| 20 | Benign | T B-25 | Normal | 0.168 | 0.150 |
| 21 | Benign | T B-70 | Normal | 0.476 | 0.068 |
| 22 | Benign | T B-77 | Normal | 0.685 | 0.164 |
| 23 | Benign | T B-79 | Normal | 0.417 | 0.213 |
| 24 | Benign | T B-80 | Normal | 1.218 | 0.204 |
| 25 | Benign | T B-81 | Normal | 0.188 | 0.126 |
| 26 | Benign | T B-82 | Normal | 0.635 | 0.832 |
| 27 | Benign | T B-83 | Normal | 0.400 | 0.380 |
| 28 | Normal | WCS-02 | Normal | 0.057 | 0.043 |
| 29 | Normal | WCS-04 | Normal | 0.297 | 0.110 |
| 30 | Normal | WCS-12 | Normal | 0.082 | 0.076 |
| 31 | Normal | WCS-13 | Normal | 0.062 | 0.034 |
| 32 | Normal | WCS-14 | Normal | 0.203 | 0.090 |
| 33 | Normal | WCS-15 | Normal | 0.113 | 0.162 |

TABLE 4B

| | CLIC1 LAALNPESN- | | PRDX6 | PRDX6 | PRDX6 |
|---|---|---|---|---|---|
| | TAGLDIFAK | CLIC1 | VVFVFGPDK | LSILYPATTGR | LPFPIIDDR |
| # | (923.1/1136.6) | Average | (504.3/809.4) | (596.3/765.4) | (543.3/728.4) |
| 1 | 0.701 | 0.747 | 0.466 | 0.841 | 0.769 |
| 2 | 1.864 | 1.780 | 0.645 | 1.281 | 1.147 |
| 3 | 0.799 | 0.810 | 0.361 | 0.738 | 0.622 |
| 4 | 0.955 | 1.190 | 0.596 | 0.856 | 0.904 |
| 5 | 0.561 | 0.505 | 0.915 | 0.410 | 0.523 |
| 6 | 2.158 | 2.320 | 0.797 | 1.248 | 1.228 |
| 7 | 1.438 | 1.600 | 7.542 | 3.226 | 3.955 |
| 8 | 0.952 | 0.980 | 0.719 | 0.393 | 0.441 |
| 9 | 0.060 | 0.063 | 0.056 | 0.352 | 0.061 |
| 10 | 0.368 | 0.336 | 0.221 | 0.507 | 0.362 |
| 11 | 2.081 | 1.749 | 2.256 | 2.540 | 3.352 |
| 12 | 1.920 | 2.280 | 1.696 | 2.254 | 2.366 |
| 13 | 1.612 | 1.565 | 0.403 | 0.728 | 0.653 |
| 14 | 0.528 | 0.404 | 0.381 | 0.956 | 0.422 |
| 15 | 1.090 | 0.848 | 0.325 | 0.512 | 0.369 |
| 16 | 0.141 | 0.197 | 0.147 | 0.410 | 0.243 |
| 17 | 0.448 | 0.403 | 0.301 | 0.501 | 0.383 |
| 18 | 0.294 | 0.193 | 0.174 | 0.246 | 0.200 |
| 19 | 0.127 | 0.208 | 0.227 | 0.331 | 0.213 |
| 20 | 0.147 | 0.155 | 0.133 | 0.419 | 0.303 |
| 21 | 0.341 | 0.295 | 0.406 | 0.521 | 0.623 |
| 22 | 0.609 | 0.486 | 0.359 | 0.850 | 0.586 |
| 23 | 0.435 | 0.355 | 0.203 | 0.429 | 0.262 |
| 24 | 1.091 | 0.837 | 0.328 | 0.378 | 0.498 |
| 25 | 0.154 | 0.156 | 0.117 | 0.121 | 0.106 |
| 26 | 1.579 | 1.015 | 1.416 | 20.48 | 2.160 |
| 27 | 0.475 | 0.418 | 0.593 | 0.945 | 1.021 |
| 28 | 0.161 | 0.087 | 0.120 | 0.436 | 0.142 |
| 29 | 0.341 | 0.250 | 0.070 | 0.265 | 0.055 |
| 30 | 0.053 | 0.070 | 0.050 | 0.185 | 0.055 |
| 31 | 0.060 | 0.052 | 0.021 | 0.236 | 0.044 |
| 32 | 0.174 | 0.156 | 0.262 | 0.720 | 0.557 |
| 33 | 0.261 | 0.179 | 0.165 | 0.321 | 0.184 |

TABLE 4C

| | | CTSD-30 kDa | CTSD-30 kDa | |
|---|---|---|---|---|
| | PRDX6 | QVFGEATK | VGFAEAAR | CTSD-30 kDa |
| # | Average | (440.2/652.2) | (410.7/721.4) | Average |
| 1 | 0.692 | 1.998 | 2.135 | 2.066 |
| 2 | 1.024 | 2.631 | 2.541 | 2.586 |
| 3 | 0.574 | 0.409 | 0.447 | 0.428 |
| 4 | 0.785 | 0.673 | 0.700 | 0.686 |
| 5 | 0.616 | 1.044 | 0.799 | 0.921 |
| 6 | 1.091 | 1.618 | 1.469 | 1.544 |
| 7 | 4.908 | 0.954 | 0.694 | 0.824 |
| 8 | 0.518 | 1.251 | 1.493 | 1.372 |
| 9 | 0.156 | 0.506 | 0.641 | 0.573 |
| 10 | 0.364 | 0.194 | 0.303 | 0.249 |
| 11 | 2.716 | 1.218 | 1.420 | 1.319 |
| 12 | 2.105 | 1.429 | 1.246 | 1.338 |
| 13 | 0.594 | 1.831 | 1.817 | 1.824 |
| 14 | 0.586 | 0.672 | 0.631 | 0.651 |
| 15 | 0.402 | 0.720 | 0.594 | 0.657 |
| 16 | 0.267 | 0.079 | 0.219 | 0.149 |
| 17 | 0.395 | 0.444 | 0.410 | 0.427 |
| 18 | 0.207 | 0.330 | 0.442 | 0.386 |
| 19 | 0.257 | 0.355 | 0.351 | 0.353 |
| 20 | 0.285 | 0.371 | 0.493 | 0.432 |
| 21 | 0.517 | 0.163 | 0.081 | 0.122 |
| 22 | 0.598 | 0.110 | 0.131 | 0.120 |
| 23 | 0.298 | 0.234 | 0.344 | 0.289 |
| 24 | 0.401 | 0.335 | 0.362 | 0.349 |
| 25 | 0.114 | 0.305 | 0.248 | 0.276 |
| 26 | 1.875 | 0.143 | 0.207 | 0.175 |
| 27 | 0.853 | 0.129 | 0.297 | 0.213 |
| 28 | 0.233 | 0.679 | 0.600 | 0.639 |
| 29 | 0.130 | 0.382 | 0.359 | 0.370 |
| 30 | 0.097 | 0.849 | 0.645 | 0.747 |
| 31 | 0.101 | 0.402 | 0.337 | 0.369 |
| 32 | 0.513 | 0.234 | 0.175 | 0.204 |
| 33 | 0.223 | 0.708 | 0.744 | 0.726 |

As further stated above, the biomarkers/biomarker signatures described above, can in another embodiment, refer to nucleic acid sequences, genes and transcripts encoding the biomarkers and expression profiles thereof.

### III. DIAGNOSTIC REAGENTS, DEVICES AND KITS

a. Labeled or Immobilized Biomarkers or Peptides or Molecular Forms

In one embodiment, diagnostic reagents or devices for use in the methods of diagnosing ovarian cancer include one or more target biomarker or peptide fragment identified in Table 1 herein, including, but not limited to, peptides identified in Table 3, or molecular forms thereof, associated with a detectable label or portion of a detectable label system. In another embodiment, a diagnostic reagent includes one or more target biomarker or peptide fragment thereof identified in Table 1, including but not limited to the peptides of Table 3 herein, immobilized on a substrate. In still another embodiment, combinations of such labeled or immobilized biomarkers are suitable reagents and components of a diagnostic kit or device.

In another aspect, suitable embodiments of such labeled or immobilized reagents include at least one, 2, 3, 4, 5, 6, 7, 8, 9, 10 or all 11 of biomarkers of Table 1 or their unique peptide fragments therein (see Table 3).

In one aspect the reagent, device or kit comprises or consists of ligands that individually specifically complex with, bind to, or quantitatively detect or identify multiple isoforms of any of biomarkers (a) through (l).

In another aspect, the reagent, device or kit comprises or consists of ligands that individually specifically complex with, bind to, or quantitatively detect or identify two or more biomarkers selected from CTSD-30 kDa, CLIC1, PRDX6, TPM1, BPGM and PSMA7, or an isoform, pro-form, modified molecular form, or peptide fragment thereof.

In another aspect, the reagent, device or kit comprises or consists of ligands that individually specifically complex with, bind to, or quantitatively detect or identify two or more biomarkers selected from CTSD-30 kDa, CLIC1 and PRDX6, or an isoform, pro-form, modified molecular form, or peptide fragment thereof.

In another aspect, the reagent, device or kit comprises or consists of ligands that individually specifically complex with, bind to, or quantitatively detect or identify two or more biomarkers selected from any of the specific combinations of biomarkers recited above.

In another embodiment suitable embodiments include at least one biomarker, CA-125, or an isoform, pro-form, modified molecular form, or peptide fragment thereof.

Still other diagnostic reagents are the surrogate peptides used for the MRM assays, such as, but not limited to, the peptides disclosed in Table 3.

Any combination of labeled or immobilized biomarkers can be assembled in a diagnostic kit or device for the purposes of diagnosing ovarian cancer, such as those combinations of biomarkers discussed herein.

For these reagents, the labels may be selected from among many known diagnostic labels, including those described above. Similarly, the substrates for immobilization in a device may be any of the common substrates, glass, plastic, a microarray, a microfluidics card, a chip, a bead or a chamber.

### B. Labeled or Immobilized Ligands that Bind or Complex with the Biomarkers

In another embodiment, the diagnostic reagent or device includes a ligand that binds to or complexes with a biomarker of Table 1 or a unique peptide thereof, as indicated in Table 3 or a molecular form thereof or a combination of such ligands.

In one embodiment, such a ligand desirably binds to a protein biomarker or a unique peptide contained therein, and can be an antibody which specifically binds a single biomarker of Table 1, or a unique peptide in that single biomarker. Various forms of antibody, e.g., polyclonal, monoclonal, recombinant, chimeric, as well as fragments and components (e.g., CDRs, single chain variable regions, etc.) or antibody mimics or equivalents may be used in place of antibodies. The ligand itself may be labeled or immobilized.

In another embodiment, suitable labeled or immobilized reagents include at least 2, 3, 4, 5, 6, 7 8, 9, 10 or 11 or more ligands. Each ligand binds to or complexes with a single biomarker protein/peptide, fragment, or molecular form of the biomarker(s) of Table 1 or peptide/peptide encoding sequence of Table 3. In some of the embodiments in which the combination of biomarkers includes CA125 or another known additional biomarker that may be in higher abundance in serum, ligands to each of these additional biomarkers may be employed in the diagnostic reagent.

Any combination of labeled or immobilized biomarker ligands can be assembled in a diagnostic kit or device for the purposes of diagnosing ovarian cancer.

Thus, a kit or device can contain multiple reagents or one or more individual reagents. For example, one embodiment of a composition includes a substrate upon which the biomarkers or ligands are immobilized. In another embodiment, the kit also contains optional detectable labels, immobilization substrates, optional substrates for enzymatic labels, as well as other laboratory items.

The diagnostic reagents, devices, or kits compositions based on the biomarkers or fragments selected from Tables 1 or Table 3 described herein, optionally associated with detectable labels, can be presented in the format of a microfluidics card, a chip or chamber, a bead or a kit adapted for use with assays formats such as sandwich ELISAs, multiple protein assays, platform multiplex ELISAs, such as the BioRad Luminex platform, Mass spectrometry quantitative assays, or PCR, RT-PCR or Q PCR techniques.

In one embodiment, a kit includes multiple antibodies directed to bind to one or more of the combinations of biomarkers described above, wherein the antibodies are associated with detectable labels.

In another embodiment, the reagent ligands are nucleotide sequences, the diagnostic reagent is a polynucleotide or oligonucleotide sequence that hybridizes to gene, gene fragment, gene transcript or nucleotide sequence encoding a biomarker of Table 1 or encoding a unique peptide thereof, as indicated in Table 3. Such a polynucleotide/oligonucleotide can be a probe or primer, and may itself be labeled or immobilized. In one embodiment, ligand-hybridizing polynucleotide or oligonucleotide reagent(s) are part of a primer-probe set, and the kit comprises both primer and probe. Each said primer-probe set amplifies a different gene, gene fragment or gene expression product that encodes a different biomarker of Table 1, optionally including one or more additional known biomarkers, such as CA125. For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publically available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, optimal PCR primers and probes used in the compositions described herein are generally 17-30 bases in length, and contain about 20-80%, such as, for example, about 50-60% G+C bases. Melting temperatures of between 50 and 80° C., e.g. about 50 to 70° C. are typically preferred.

The selection of the ligands, biomarker sequences, their length, suitable labels and substrates used in the reagents and kits are routine determinations made by one of skill in the art in view of the teachings herein of which biomarkers form signature suitable for the diagnosis of ovarian cancer.

### IV. METHODS FOR DIAGNOSING OR MONITORING OVARIAN CANCER

In another embodiment, a method for diagnosing or detecting or monitoring the progress of ovarian cancer in a subject comprises, or consists of, a variety of steps.

### A. Sample Preparation

The test sample is obtained from a human subject who is to undergo the treatment. The subject's sample can in one embodiment be provided before initial diagnosis, so that the method is performed to diagnose the existence of an ovarian cancer. In another embodiment, depending upon the reference standard and markers used, the method is performed to diagnosis the stage of ovarian cancer. In another embodiment, depending upon the reference standard and markers used, the method is performed to diagnosis the type or subtype of ovarian cancer from the types and subtypes identified above. In another embodiment, the subject's sample can be provided after a diagnosis, so that the method is performed to monitor progression of an ovarian cancer. In another embodiment, the sample can be provided prior to surgical removal of an ovarian tumor or prior to therapeutic treatment of a diagnosed ovarian cancer and the method used to thereafter monitor the effect of the treatment or surgery, and to check for relapse. In another embodiment, the sample can be provided following surgical removal of an ovarian tumor or following therapeutic treatment of a diagnosed ovarian cancer, and the method performed to ascertain efficacy of treatment or relapse. In yet another embodiment the sample may be obtained from the subject periodically during therapeutic treatment for an ovarian cancer, and the method employed to track efficacy of therapy or relapse. In yet another embodiment the sample may be obtained from the subject periodically during therapeutic treatment to enable the physician to change therapies or adjust dosages. In one or more of these embodiments, the subject's own prior sample can be employed in the method as the reference standard.

Preferably where the sample is a fluid, e.g., blood, serum or plasma, obtaining the sample involves simply withdrawing and preparing the sample in traditional fashion for contact with the diagnostic reagent. Where the sample is a tissue or tumor sample, it may be prepared in conventional manner for contact with the diagnostic reagent.

The method further involves contacting the sample obtained from a test subject with a diagnostic reagent as described above under conditions that permit the reagent to bind to or complex with one or more biomarker(s) of Table 1 which may be present in the sample. This method may employ any of the suitable diagnostic reagents or kits or compositions described above.

B. Measuring Biomarker Levels

Thereafter, a suitable assay is employed to detect or measure in the sample the protein level (actual or relative) of one or more biomarker(s) of Table 1. Alternatively, a suitable assay is employed to generate a protein abundance profile (actual or relative or ratios thereof) of multiple biomarkers of Table 1 from the sample or of multiple different molecular forms of the same biomarker or both. In another embodiment, the above method further includes measuring in the biological sample of the subject the protein level of an additional biomarker, such as CA125 or other known ovarian cancer biomarker. In another embodiment, the above method further includes measuring in the biological sample of the subject the protein levels of two or more additional biomarkers which form a biomarker protein abundance signature for ovarian cancer.

The measurement of the biomarker(s) in the biological sample may employ any suitable ligand, e.g., antibody, antibody mimic or equivalent (or antibody to any second biomarker) to detect the biomarker protein. Such antibodies may be presently extant in the art or presently used commercially, such as those available as part of commercial antibody sandwich ELISA assay kits or that may be developed by techniques now common in the field of immunology. A recombinant molecule bearing the binding portion of a biomarker antibody, e.g., carrying one or more variable chain CDR sequences that bind e.g., PRDX6, CTSD-30, CDSD-52, CLIC1, etc. may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers of Table 1 or a particular sequence of the selected biomarker as defined in Table 1 or Table 3 may be employed in the methods described herein, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods and devices of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays and devices of this invention may be easily selected from among numerous compositions known and readily available to one skilled in the art of diagnostic assays. The biomarker-antibodies or fragments useful in this invention are not limited by the particular detectable label or label system employed. Thus, selection and/or generation of suitable biomarker antibodies with optional labels for use in this invention is within the skill of the art, provided with this specification, the documents incorporated herein, and the conventional teachings of immunology.

Similarly the particular assay format used to measure the selected biomarker in a biological sample may be selected from among a wide range of protein assays, such as described in the examples below. Suitable assays include enzyme-linked immunoassays, sandwich immunoassays, homogeneous assays, immunohistochemistry formats, or other conventional assay formats. In one embodiment, a serum/plasma sandwich ELISA is employed in the method. In another embodiment, a mass spectrometry-based assay is employed. In another embodiment, a MRM assay is employed, in which antibodies are used to enrich the biomarker in a manner analogous to the capture antibody in sandwich ELISAs.

One of skill in the art may readily select from any number of conventional immunoassay formats to perform this invention.

Other reagents for the detection of protein in biological samples, such as peptide mimetics, synthetic chemical compounds capable of detecting the selected biomarker may be used in other assay formats for the quantitative detection of biomarker protein in biological samples, such as high pressure liquid chromatography (HPLC), immunohistochemistry, etc.

Employing ligand binding to the biomarker proteins or multiple biomarkers forming the signature enables more precise quantitative assays, as illustrated by the multiple reaction monitoring (MRM) mass spectrometry (MS) assays. As an alternative to specific peptide-based MRM-MS assays that can distinguish specific protein isoforms and proteolytic fragments, the knowledge of specific molecular forms of biomarkers allows more accurate antibody-based assays, such as sandwich ELISA assays or their equivalent. Frequently, the isoform specificity and the protein domain specificity of immune reagents used in pre-clinical (and some clinical) diagnostic tests are not well defined. MRM-MS assays were used to quantitative the levels of a number of the low abundance biomarkers in samples, as discussed in the examples.

In one embodiment, suitable assays for use in these methods include immunoassays using antibodies or ligands to the above-identified biomarkers and biomarker signatures. In another embodiment, a suitable assay includes a multiplexed MRM based assay for two more biomarkers that include one or more of the proteins/unique peptides in Table 1 and Table 3. It is anticipated that ultimately the platform most likely to be used in clinical assays will be multiplexed or parallel sandwich ELISA assays or their equivalent, primarily because this platform is the technology most commonly used to quantify blood proteins in clinical laboratories. MRM MS assays may continue to be used productively to help evaluate the isoform/molecular form specificity of any existing immunoassays or those developed in the future.

### C. Detection of a Change in Biomarker Abundance Level and Diagnosis

The protein level of the one or more biomarker(s) in the subject's sample or the protein abundance profile of multiple said biomarkers as detected by the use of the assays described above is then compared with the level of the same biomarker or biomarkers in a reference standard or reference profile. In one embodiment, the comparing step of the method is performed by a computer processor or computer-programmed instrument that generates numerical or graphical data useful in the appropriate diagnosis of the condition. Optionally, the comparison may be performed manually.

The detection or observation of a change in the protein level of a biomarker or biomarkers in the subject's sample from the same biomarker or biomarkers in the reference standard can indicate an appropriate diagnosis. An appropriate diagnosis can be identifying a risk of developing ovarian cancer, a diagnosis of ovarian cancer (or stage or type thereof), a diagnosis or detection of the status of progression or remission of ovarian cancer in the subject following therapy or surgery, a determination of the need for a change in therapy or dosage of therapeutic agent. The method is thus useful for early diagnosis of disease, for monitoring response or relapse after initial diagnosis and treatment or to predict clinical outcome or determine the best clinical treatment for the subject.

In one embodiment, the change in protein level of each biomarker can involve an increase of a biomarker or multiple biomarkers in comparison to the specific reference standard. In one embodiment, the biomarkers of Table 1, e.g., CTSD and CLIC1, are increased in a subject sample from a patient having ovarian cancer when compared to the levels of these biomarkers from a healthy reference standard. In another embodiment, the biomarkers of Table 1, e.g., CTSD and CLIC1, are increased in a subject sample from a patient having ovarian cancer prior to therapy or surgery, when compared to the levels of these biomarkers from a post-surgery or post-therapy reference standard.

In another embodiment, the change in protein level of each biomarker can involve a decrease of a biomarker or multiple biomarkers in comparison to the specific reference standard. In one embodiment, the biomarkers of Table 1, e.g., CTSD and CLIC1, are decreased in a subject sample from a patient having ovarian cancer following surgical removal of a tumor or following chemotherapy/radiation when compared to the levels of these biomarkers from a pre-surgery/pre-therapy ovarian cancer reference standard or a reference standard which is a sample obtained from the same subject pre-surgery or pre-therapy.

In still other embodiments, the changes in protein levels of the biomarkers may be altered in characteristic ways if the reference standard is a particular type of ovarian cancer, e.g., serous, epithelial, mucinous or clear cell, or if the reference standard is derived from benign ovarian cysts or nodules.

The results of the methods and use of the compositions described herein may be used in conjunction with clinical risk factors to help physicians make more accurate decisions about how to manage patients with ovarian cancers. Another advantage of these methods and compositions is that diagnosis may occur earlier than with more invasive diagnostic measures.

### D. Alternative Embodiments

In an alternative embodiment, the method of diagnosis or risk of diagnosis involves using the nucleic acid hybridizing reagent ligands described above to detect a significant change in expression level of the subject's sample biomarker or biomarkers from that in a reference standard or reference expression profile which indicates a diagnosis, risk, or the status of progression or remission of ovarian cancer in the subject. These methods may be performed in other biological samples, e.g., biopsy tissue samples, tissue removed by surgery, or tumor cell samples, including circulating tumor cells isolated from the blood, to detect or analyze a risk of developing an ovarian cancer, as well as a diagnosis of same. Such methods are also known in the art and include contacting a sample obtained from a test subject with a diagnostic reagent comprising a ligand which is a nucleotide sequence capable of hybridizing to a nucleic acid sequence encoding a biomarker of Table 1, said ligand associated with a detectable label or with a substrate. Thereafter one would detect or measure in the sample or from an expression profile generated from the sample, the expression levels of one or more of the biomarkers or ratios thereof. The expression level(s) of the biomarker(s) in the subject's sample or from an expression profile or ratio of multiple said biomarkers are then compared with the expression level of the same biomarker or biomarkers in a reference standard. A significant change in expression level of the subject's sample biomarker or biomarkers from that in the reference standard indicates a diagnosis, risk, or the status of progression or remission of ovarian cancer in the subject.

Suitable assay methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The comparison of the quantitative or relative expression levels of the biomarkers may be done analogously to that described above for the comparison of protein levels of biomarkers.

### E. Non-Ligand-Based Analysis

In another aspect, a method for diagnosing or detecting or monitoring the progress of ovarian cancer in a subject involves non-ligand based methods, such as mass spectrometry. For example, proteins in a biological sample obtained from a test subject may be contacted with a chemical or enzymatic agent and the proteins, including the biomarkers contained therein fragmented in the sample. The digested sample or portions thereof are injected into a mass spectrometer and the protein levels or ratios of one or more of the biomarkers of Table 1, optionally with other known biomarkers, modified molecular forms, peptides and unique peptides or ratios thereof, are quantitatively identified or measured by mass spectrometry. The protein levels of the biomarkers in the subject's sample are then compared with the level of the same biomarker or biomarkers in a reference standard or to a predetermined cutoff derived from the reference standard. In one embodiment, the agent is a proteolytic enzyme. In another embodiment, the agent is trypsin.

A significant change in protein level of the subject's sample biomarker or biomarkers from that in the reference standard or from a predetermined cutoff indicates a diagnosis, risk, or the status of progression or remission of ovarian cancer in the subject.

Thus, the various methods, devices and steps described above can be utilized in an initial diagnosis of ovarian cancer or other ovarian condition, as well as in clinical management of patients with ovarian cancer after initial diagnosis. Uses in clinical management of the various devices, reagents and assay methods, include without limitation, monitoring for reoccurrence of disease or monitoring remission or progression of the cancer and either before, during or after therapeutic or surgical intervention, selecting among therapeutic protocols for individual patients, monitoring for development of toxicity or other complications of therapy, and predicting development of therapeutic resistance.

In one embodiment, the method involves enriching the biomarker protein or one or more peptides produced by specific proteolysis in the sample by contacting the sample with an antibody prior to injecting into a mass spectrometer in a manner analogous to a capture antibody in a conventional sandwich ELISA. In another embodiment, the method involves depleting the sample of non-target proteins prior to injecting sample into a mass spectrometer. The depletion may also be performed using antibodies to the non-targets. The method described herein may use liquid chromatographic mass spectrometry, such as HPLC. One such method is described in detail in the Examples below.

### V. EXAMPLES

Human blood, in the form of plasma or serum, is one of the most valuable specimens for protein biomarker discovery because it is routinely collected, collection is minimally invasive, and it contains thousands of proteins, including those secreted or shed into the blood by tumors.²⁰ However, systematic discovery of serological biomarkers directly from human serum using proteomics¹⁹ has proven extremely challenging due to the extremely wide concentration range of blood proteins that span more than 10 orders of magnitude. In addition, the most tumor-specific proteins are very likely to primarily be shed by the tumor and will be very low abundant in blood, as exemplified by well-known cancer biomarkers such as PSA and CEA, which are present in serum in the low ng/ml to pg/ml range.^{20, 21} Most cancers and other diseases also elicit a wide range of host response mechanisms, producing many acute-phase or inflammation-related proteins. It is unlikely that most such relatively general host responses will have sufficient specificity and sensitivity for cancer detection in at-risk populations, although selected inflammation-related biomarkers could contribute to panels of biomarkers that include proteins specifically shed by the tumor. Regardless, it is clear that these common, acute-phase-related changes in serum proteins hamper discovery of tumor-specific proteins when directly profiling sera in human populations. Finally, individual protein levels in blood are highly variable in the human population due to extensive genetic, physiological, and environmental variations, requiring analysis of many patient and control samples before statistically significant, disease-related differences can be identified.

The inventors determined that dynamic range and complexity of the blood proteome can be addressed by major protein depletion and multidimensional sample prefractionation. We and others have shown that multidimensional sample prefractionation prior to mass spectrometry analysis greatly enhances proteome coverage and allows detection of low-abundance proteins, at least down to the low ng/ml range.²²⁻²⁷ To overcome the genetic, physiological, and environmental variability associated with analyzing human samples, many less complex experimental models, including cancer cell lines in culture,^{28, 29} cancer tissue specimens,^{30, 31} ascites fluid,^{32, 33} secretomes,^{34, 35} and mouse models,³⁶⁻³⁸ have been used in ovarian cancer biomarker discovery. Each model has its benefits, but most strategies, except for the use of mouse models, are not able to determine if the discovered biomarkers are actually shed into blood. In ovarian cancer, the use of both genetically engineered and xenograft mouse models to facilitate serum biomarker discovery has been described.³⁶⁻³⁸ Even though subject-to-subject heterogeneity is considerably reduced with the use of genetically engineered mouse models, these models still produce many host-response protein changes that can be difficult to distinguish from more tumor-specific protein changes.³⁷

The use of xenograft mouse models has several advantages over other models. First, proteins shed by human tumors into mouse blood can be unambiguously distinguished from less-specific host responses by exploiting species differences in peptide sequences identified by liquid chromatography-tandem mass spectrometry (LC-MS/MS). Second, the blood volume of a mouse is approximately 5,000 times less than an adult human. Therefore, proteins shed by similar-sized small tumors in a mouse and an adult human are likely to be at least 1,000 times more concentrated in a xenograft mouse as compared to the same size tumor in a human. Third, the minimal biological heterogeneity of the xenograft mouse model means that only a small number of samples need to be profiled in order to make inferences about putative biomarkers.

While the use of xenograft mouse models potentially can improve detection of novel cancer biomarkers, mouse serum is still a very complex proteome and requires multidimensional sample prefractionation for sufficient depth of analysis. For example, in a prior xenograft mouse study using two-dimensional gel electrophoresis and without any sample prefractionation, only acute-phase proteins were identified successfully.³⁹ In the case of ovarian cancer, two different studies using a xenograft model with human SKOV-3 serous ovarian cancer cells have been described. In one study, mouse sera were trypsin-digested and analyzed directly by LC-MS/MS, resulting in identification of 13 human proteins.³⁸ The other study focused on the low molecular weight serum proteome/peptidome of the xenograft model and reported the identification of five human proteins.³⁶

As demonstrated in the examples below, the inventors combined a xenograft mouse model using the ovarian endometrioid TOV-112D cell line with a higher performance, multidimensional prefractionation strategy. We analyzed the serum proteome using an in-depth 4-D protein profiling method to identify human proteins in the mouse serum. Identified putative biomarkers are shed by the tumor and we detected many tumor-derived human proteins, including low-abundance human proteins that are present at <100 ng/ml in normal human serum. Such difficult-to-detect proteins, which are in lower abundance in the serum, are anticipated to be more tumor-specific. Detection of low-abundance proteins shed by the tumor is enhanced because the mouse blood volume is more than a thousand times smaller than that of a human. Using TOV-112D ovarian tumors, more than 200 human proteins were identified in the mouse serum, including candidate biomarkers and proteins previously reported to be elevated in either ovarian tumors or the blood of ovarian cancer patients. These proteins included some previously known ovarian cancer-associated proteins. Subsequent quantitation of selected biomarkers was conducted in human sera from ovarian patients, as well as normal controls and patients with benign disease, using label-free multiple reaction monitoring (MRM) mass spectrometry (MS).

A substantial portion of the indicated biomarkers correlate with ovarian cancer in patient serum specimens. Quantitation of several candidate biomarkers in ovarian patient sera identified the biomarkers in Table 1, as ovarian cancer biomarkers. For example, chloride intracellular channel 1, the mature form of cathepsin D, and peroxiredoxin 6 were elevated significantly in sera from ovarian carcinoma patients. Table 2 illustrates differences between the abundant biomarkers and the biomarkers considered useful in the present compositions and methods.

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1

### Materials and Methods

Cell Culture.

The human epithelial ovarian cancer cell line TOV-112D was obtained from the American Type Culture Collection (ATCC, Manassas, Va.). The TOV-112D cells were maintained in a 37° C. incubator with a 5% CO₂-95% air atmosphere in a 1:1 mixture of MCDB 105 media and Medium 199 (Sigma-Aldrich, St. Louis, Mo.) supplemented with 15% fetal calf serum, as described previously.⁴⁰

Ovarian Cancer Growth In Vivo and Mouse Serum Collection.

Approximately 3 million TOV-112D cells in 100 μl PBS were injected subcutaneously into the flanks of nine severe combined immunodeficient (SOD) female mice. Tumor volumes were monitored by caliper measurements. When tumors were approximately 1 cm in length, blood was collected from mice by cardiac puncture under anesthesia, animals were immediately euthanized, and the tumor at the injection site and other internal organs were collected.

The collected blood was allowed to clot at room temperature and followed by centrifugation for 15 min at 4° C. to collect the serum. Individual aliquots of serum from each mouse were then snap-frozen and stored at −80° C. Serum subsequently was thawed briefly and pooled based on assessment of tumor burden and extent of tumor necrosis. The total protein concentrations of pooled serum samples were measured using a BCA Protein Assay (Pierce Chemical, Rockford, Ill.), after which the pooled serum was re-aliquoted, snap-frozen, and stored at −80° C. until future use. Tumor necrosis was assessed by microscopic inspection of hematoxylin and eosin (H&E) stained paraffin-embedded sections (5 μm), and other organs were macroscopically and microscopically examined for evidence of tumor metastasis.

Immunoaffinity Removal of Major Mouse Serum Proteins.

The pooled mouse serum was depleted using a 4.6×100 mm MARS Mouse-3 HPLC column (Agilent Technologies, Wilmington, Del.). A total of 225 μL pooled serum was diluted five-fold with the manufacturer's equilibration buffer, filtered through a 0.22 μm microcentrifuge filter, and briefly stored on ice. This sample subsequently was applied to the antibody column in five serial injections of 200-250 μl per depletion. The flow-through fractions containing unbound proteins were collected and pooled. The immunodepletion equilibration buffer was removed by buffer exchange into 10 mM sodium phosphate, pH 7.0, and the sample was concentrated to the initial serum volume using a 5K molecular weight cutoff (MWCO) spin concentrator. Bound proteins were eluted with the manufacturer's elution buffer, neutralized with 1 M NaOH, pooled, aliquoted, and stored at −20° C. for possible future analysis. Protein concentrations from the unbound and bound fractions were estimated using standard and reducing-reagent-compatible BCA assays, respectively.

MicroSol-IEF Fractionation.

Depleted and concentrated mouse serum (2.6 mg) was reduced with 20 mM DTT for 30 mM and alkylated with 50 mM N,N-dimethylacrylamide (DMA) for 30 min at room temperature in 550 μl of buffer (final volume) containing 8 M urea, 20 mM Tris-HCL, pH 8.5. Alkylation was quenched with 1% DTT, and serum was diluted to 670 μl (final volume) in a sample buffer consisting of 8 M urea, 2 M thiourea, 4% CHAPS, 1% DTT, 1% pH 3-7 ZOOM focusing buffer, and 1% pH 7-12 ZOOM focusing buffer. Serum was fractionated by MicroSol-IEF, as previously described^{23, 41} using a ZOOM-IEF fractionator (Invitrogen, Carlsbad, Calif.), into five, small-volume (550-650 μL) pools where the separation chambers were defined by IPG gel membranes having pH values of 3.0, 4.6, 5.4, 6.2, and 7.0, respectively. After focusing, samples were removed and each chamber was rinsed with a small volume of sample buffer, which was combined with the solution removed from that chamber. IPG gel membranes were extracted twice for 1 h each with 100 μL of 1% SDS, 20 mM Tris, 1% 2-mercaptoethanol. To maximize protein loads for SDS-PAGE, MicroSol-IEF fractions were precipitated overnight with nine volumes of 200 proof ethanol, prechilled to −20° C. Ethanol supernatants were carefully removed and protein pellets were re-suspended in 50% ethanol, centrifuged, and pellets were frozen and stored at −20° C. until further use. Membrane extracts were concentrated to approximately 50 μL with 5K MWCO spin concentrators.

SDS-PAGE/in-Gel Trypsin Digestion of Mouse Serum.

Frozen protein pellets from ethanol precipitation of MicroSol IEF fractions were thawed briefly and resuspended in SDS gel sample buffer. For fractions 2-4, aliquots derived from 15 μL of original serum per lane were loaded into 10-well 12% NuPAGE mini-gels (Invitrogen) and separated using MES running buffer until the tracking dye had migrated 4 cm. For fractions 1 and 5 and for membrane extractions, the equivalent of 37 μL and 80 μL of original serum, respectively, was loaded and separated for 1 cm. Gels were stained with Colloidal Blue (Invitrogen), and each lane subsequently was sliced into either 40 (fractions 2-4) or 10 (fractions 1, 5, and membranes) uniform 1 mm slices using a disposable gel cutter (The Gel Company, San Francisco, Calif.). For fractions 2-4, two adjacent slices in a single lane were combined in a digestion well. Slices from duplicate lanes of fractions 1, 5, and membrane extractions were combined and all samples were digested overnight using 0.02 μg/μL modified trypsin (Promega, Madison, Wis.). A total of 140 digests were performed from the five IEF fractions and six membrane extracts.

LC-MS/MS.

Tryptic digests were analyzed on an LTQ-FT hybrid mass spectrometer (Thermo Electron, San Jose, Calif.) coupled with a NanoLC pump (Eksigent Technologies, Livermore, Calif.) and autosampler. Tryptic peptides were separated as described previously²² by RP-HPLC on a PicoFrit column (75 μm i.d., 15 nm tip opening; New Objective, Woburn, Mass.), packed with 8 cm of Hypersil C18 1.9-μm resin (Thermo Electron). Eluted peptides were analyzed by the mass spectrometer set to repetitively scan m/z from 400 to 1600 followed by data-dependant MS/MS scans on the six most abundant ions with dynamic exclusion enabled.

Data Analyses.

Peptides from each LC-MS/MS run were interpreted from MS/MS spectra using SEQUEST in Bioworks 3.2 (Thermo Electron). DTA files were created and searched against a combined mouse and human database generated from Uniprot (May 16, 2006), National Center for Biotechnology Information non-redundant (Feb. 5, 2006), and International Protein Index (version 3.17) databases. This composite database also contained the reversed sequences of each entry appended to the beginning of the forward database. The database was indexed with the following parameters: monoisotopic mass range of 750 to 3500, length of 4 to 100, partial tryptic cleavages with a maximum of two internal missed cleavage sites, static modification of Cys by dimethylacrylamide (+99.0684 Da) and dynamic modification of Met to methionine sulfoxide (+15.9946 Da). The DTA files were searched with a 2.5 Da peptide mass tolerance. Other search parameters were identical to those used for database indexing.

Outputs from all SEQUEST searches were combined, filtered using in-house scripts, and grouped into non-redundant proteins using DTASelect version 1.9.⁴² An in-house script was used to correct the wrong peptide m/z assignments to the C13 peaks. Peptides were filtered using mass accuracy ≦8 ppm, Sf≧0.4 and requiring full tryptic specificity for all identified peptides. This filtering scheme resulted in 1.4% peptide false positives calculated as the number of unique reversed-sequence hits/number of unique forward sequence hits. Keratin identifications were removed from the datasets as probable contaminations. Additional Java scripts were developed to compress the non-redundant protein identifications reported by DTASelect into the smallest sets of unique proteins. Peptide counts were derived after collapsing different forms (charge states and modifications) of the same peptide into a single hit. Further reduction was applied by allowing a peptide to be assigned only once to the protein that had the highest sequence coverage.

Custom software also was developed to separate mouse and human proteins based on their sequence identifiers. Putative human peptides then were searched using BLAST against a mouse-only database from Uniprot (11/2007) to remove any putative human sequences that exactly matched mouse sequences.

Human Serum Collection.

Sera from patients with benign ovarian tumors (n=9), and from late-stage ovarian cancer patients (stages III, n=15; or IV, n=3) were collected, at the time of diagnosis. Control serum samples (n=6) were collected from healthy, post-menopausal female donors. All specimens were processed in compliance with institutional review board and Health Insurance Portability and Accountability Act (HIPAA) requirements.

Processing of Human Serum Samples for MRM Analysis.

Control and patient serum samples were analyzed either individually or as pools, as follows. Samples were depleted of 20 abundant serum proteins using a ProteoPrep20 Immunodepletion Column (Sigma-Aldrich). Typically, 30-50 μL of serum was depleted, concentrated, and prepared for SDS-PAGE, as previously described.⁴³ SDS-PAGE conditions for human serum were identical to those described above for the analysis of mouse serum, with the exception that the equivalent of 10 pit of original serum were loaded into three adjacent lanes and separated for 4 cm. Gels were sliced and digested, as previously described.⁴³

Label-Free Multiple Reaction Monitoring.

MRM experiments were performed on a 4000 QTRAP hybrid triple quadrupole/linear ion trap mass spectrometer (AB Sciex, Foster City, Calif.) interfaced with a NanoACQUITY UPLC system (Waters, Milford, Mass.). Five μl of tryptic digests were injected using the partial loop injection mode onto a UPLC Symmetry trap column (180 μm i.d.×2 cm packed with 5 μm C18 resin; Waters) and then separated by RP-HPLC on a PicoFrit column (75-μm i.d., 15-μm tip opening) packed in-house with 25 cm of Magic C18 3-μm reversed-phase resin (Michrom Bioresources, Auburn, Calif.). Chromatography was performed with Solvent A (Milli-Q [Millipore, Billerica, Mass.] water with 0.1% formic acid) and Solvent B (acetonitrile with 0.1% formic acid). Peptides were eluted at 200 mL/min for 3-28% B over 42 min, 28-50% B over 26 min, 50-80% B over 5 min, 80% B for 4.5 min before returning to 3% B over 0.5 min. To minimize sample carryover, a fast blank gradient was run between each sample. An identical reference sample was run at the beginning of each set of samples and was used to normalize variation in MRM signals caused by changes in performance of the HPLC, reverse-phase column or mass spectrometer.

MRM data were acquired with a spray voltage of 2,500 V, curtain gas of 20 p.s.i., nebulizer gas of 10 p.s.i., interface heater temperature of 150° C., and a pause time of 5 ms. Multiple MRM transitions were monitored using unit resolution in both Q1 and Q3 quadrupoles to maximize specificity. Each MRM transition had a minimum dwell time of 15 s. Data analyses were performed using MultiQuant version 1.1 software (AB Sciex). The most abundant transition for each peptide was used for quantitation unless interference from the matrix was observed. In these cases, another transition free of interference was chosen for quantitation.

Statistical Analyses.

Serum levels of candidate biomarkers were compared across patient groups using a standard statistical test, such as the Mann-Whitney test for calculating p values or an unpaired, two-tailed Student's t-test. Welch's correction was applied to the t-test when the variances between the two sets were significantly different. Statistical significance was determined if the P-value of the test was less than 0.05. Calculations, scatter plots, and receiver operator characteristic (ROC) curves were generated using the GraphPad Prism 5 (GraphPad, San Diego, Calif.). Optimal cut-points were obtained by identifying a threshold for each biomarker that resulted in maximum sensitivity and specificity when used to classify serum as tumor or control. Both sensitivity and specificity for each decision rule defined by biomarker-specific optimal cut-point were computed, as well as the positive and negative predictive values. The odds ratio between the group classification and the result from each decision rule from the logistic regression was used as a measure of their association. Multivariate models were fit using logistic regression analysis.

### Example 2

### Overview of the Ovarian Cancer Biomarker Discovery and Verification/Validation Strategies

The general experimental workflow we used for discovery and verification of candidate ovarian cancer protein biomarkers is shown in FIGS. 1A and 1B. For discovery of candidate human biomarkers, serum proteins obtained from SCID mice harboring human ovarian cancer tumors were subjected to a 4-D separation consisting of three sequential, and substantially orthogonal, protein separations, i.e., major protein depletion, solution IEF, and 1-D SDS-PAGE, followed by online, reversed-phase LC peptide separation prior to MS/MS analysis. We previously developed this 4-D protein profiling method for comprehensive analysis of human serum and plasma proteomes, which resulted in the most comprehensive coverage of a serum sample in the pilot phase of the Human Proteome Organization Plasma Proteome Project (HUPO PPP).^{22, 44} That study also demonstrated that 14 of the 20 proteins known at that time to be in the 1-100 ng/ml range in normal human serum could be detected. This method has the capacity to detect many low-abundance proteins.

An independent, multiplexed, targeted mass spectrometry verification strategy utilizing label-free MRM analysis was developed for this purpose (FIG. 1B). This label-free GeLC-MRM workflow is highly reproducible and is capable of quantitating proteins in serum down to approximately 200 pg/mL, as reported in Tang et al, J. Proteome Res., 10(9):4005-4017, published on line Jul. 26, 2011. This reference is incorporated by reference herein for its description of the strategy and results reported therein.⁴⁵ SDS gel fractionation can resolve different molecular weight-forms of targeted proteins and consequently permit separate quantitation of each form of the targeted proteins. Similarly, it can distinguish between closely related protein isoforms with high confidence by targeting isoform-specific peptide sequences. The label-free GeLC-MRM workflow enables rapid, sensitive, and economical initial screening of large numbers of candidate biomarkers prior to setting up stable-isotope dilution MRM assays or immunoassays.

### Example 3

### Xenograft Mouse Model of Human Ovarian Endometrioid Cancer

To identify candidate serum biomarkers using the xenograft mouse model, TOV-112D ovarian endometrioid tumor cells were injected subcutaneously. This cell line was originally derived from a patient with a malignant ovarian tumor that had never been exposed to radiation or chemotherapy.⁴⁶ This cell line was chosen because it has a fast growth rate and has been demonstrated to form tumors readily in immune-compromised mice. More importantly, the in vitro growth characteristics of the cell line mimic the aggressive clinical behavior of the cancer.⁴⁶ Previous proteomics biomarker discovery studies used human SKOV-3 serous ovarian cancer cells.^{36, 38} Certain sets of biomarkers likely characterize different types of ovarian cancer, where some proteins are common to all or multiple cancer subtypes and other proteins are specific to a single subtype.

### Example 4

### Four-Dimensional Protein Profiling of Xenograft Mouse Serum Proteome

A four-dimensional protein profiling of human ovarian TOV-112D xenograft mouse serum proteome was conducted where abundant mouse proteins are removed.

The mouse serum was first subjected to depletion of three major proteins from a total of 225 μl (10.2 mg) serum using a MARS Mouse-3 HPLC column. Following depletion, 3.1 mg of total unbound proteins were recovered. SDS-PAGE analysis of the unbound and bound fractions showed good depletion of albumin (69 kDa) and transferrin (77 kDa), as expected (data not shown; see FIG. 2(A) of U.S. provisional application No. 61/532,881 or FIG. 2(A) of Tang et al, 2012J. Proteome Res., 11:678-691 which show unfractionated serum (S), unbound fraction (U) of the MARS Mouse 3 LC-Column, and the bound fraction (B) containing the abundant serum proteins). The third protein expected to be depleted by this column, IgG, was not apparent in this experiment because SCID mice have very low levels of immunoglobulins. Following major protein depletion, the unbound proteins were separated into five pI fractions using MicroSol-IEF, and proteins with pIs identical to the pH of the MicroSol-IEF separator membranes were extracted (data not shown; see FIG. 2B of U.S. provisional application No. 61/532,881 or FIG. 2(B) of Tang et al, 2012 J. Proteome Res., 11:678-691 which is an SDS-PAGE of MicroSol-IEF fractions (F1 to F5) of depleted mouse serum, and proteins extracted from MicroSol-IEF membrane partitions (M1 to M6)). Although the total amount of protein trapped in the MicroSol-IEF membrane partitions was very low, these fractions were included in the proteome analysis to increase its comprehensiveness.

The third fractionation step utilized 1-D SDS-PAGE, and to enhance detection of low-abundance protein, MicroSol IEF fractions and membrane extracts were concentrated and the largest possible protein loads that avoided excessive band distortion were applied onto the gels. Furthermore, because trypsin digestion of large gel volumes containing low protein amounts can be inefficient with disproportionally high adsorptive losses, the length of the electrophoretic separation was adjusted based upon sample complexity and the number of fractions desired. Hence, the most complex fractions (F2 to F4) were separated for 4 cm and cut into 20×2-mm slices, while remaining, less complex fractions were separated for 1 cm and cut into 10×1-mm slices, with corresponding slices from duplicate lanes combined for trypsin digestion (data not shown; see FIG. 2(C) of U.S. provisional application No. 61/532,881 or FIG. 2(C) of Tang et al, 2012 J. Proteome Res., 11:678-691 which is a separation of MicroSol-IEF samples for in-gel trypsin digestion and LC-MS/MS analysis. Membrane (M1-M6) and solution fractions (F1-F5) were separated as indicated. Volumes indicate the original serum volumes from which the loaded fractions were derived). This yielded a total of 140 samples for trypsin digestion and LC-MS/MS analysis.

### Example 5

### Ovarian Cancer Xenograft Mouse Serum Proteome

From a total of 140 LC-MS/MS runs performed on a LTQ-FT mass spectrometer, 1.2 million MS/MS spectra were acquired and searched against a mouse and human composite database. After stringent data filtering and removal of redundant entries and common contaminants, a total of 1,198 unique proteins were identified from 6,014 unique peptides (FIG. 2). The peptide false discovery rate estimated from the number of hits against the reversed entries in the composite database was 1.4%. As expected, the majority of identified proteins (753) were mouse proteins, as they contained peptide sequences unique to the mouse database. Based on the database search results, 222 proteins were initially identified as human proteins because they contained at least one apparent human-specific peptide.

To confirm the species assignment, all peptides for these putative human proteins were searched against a mouse database using BLAST, and the results were used to remove proteins where all peptides were identical to mouse sequences or contained only isobaric differences (Ile/Leu). After the BLAST analysis, a total of 573 unique human-specific peptides remained and they defined 208 high-confidence human proteins identified by at least one human-specific peptide. The apparent molecular weights of these human proteins ranged from 10 to 435 kDa.

87% of identified human-specific peptides differed from the homologous mouse sequences by more than a single nucleotide. This result indicated that very few apparently human proteins were misidentified as human due to single nucleotide polymorphisms (SNPs) or deamidation of an asparagine to an aspartic acid. Two examples of MS/MS spectral assignments for human-specific peptides and the corresponding mouse sequences are shown in FIGS. 3A and 3B. In both examples, the identified human-specific peptide sequences differ from the homologous mouse sequences by more than one residue, unambiguously indicating that the identified proteins had to be secreted by the human ovarian tumors into the mouse blood. In addition to the mouse or human-specific proteins, 237 proteins were identified by peptides common to both mouse and human sequences, and are therefore species indistinguishable at this stage.

These results demonstrate the success of identifying large numbers of human-specific proteins from xenograft mouse models of solid tumor cancers when an in-depth analysis of the serum proteome is performed using a 4-D protein profiling strategy. The most specific cancer biomarkers are very low-abundance proteins that are only shed by the tumor. Proteins identified by single peptides are likely to be among the lowest abundance proteins in these datasets because there is a rough correlation between protein abundance and the number of peptides identified. Even if all single-peptide proteins were disregarded, 106 human proteins were identified by at least two peptides.

Previous serum proteomics studies of ovarian cancer xenograft mouse models only identified a few human-specific proteins, presumably due to lower levels of sample fractionation used in those studies.^{36, 38} In the study that analyzed the low molecular weight serum proteome using LC-MS/MS analysis, only 400 peptides were identified, and these peptides corresponded to a total combination of 300 human and mouse proteins. By using MS/MS spectral counts, five human-specific proteins were identified at a higher level in the cancer versus control xenograft model.³⁶ Another study directly analyzed the xenograft serum proteome by LC-MS/MS and identified 13 human proteins. However, most of the human species assignments were made by comparing the results with those obtained from a separately analyzed SKOV-3 cell line secretome.³⁸ In fact, the candidate biomarker reported in that study (14-3-3 Zeta) was identified by a single peptide that is indistinguishable from the corresponding mouse sequence. Proteomics profiling of xenograft mouse models of prostate, breast, and oral squamous cell carcinomas also has been reported.^{47, 49} These studies utilized three or less dimensions of sample fractionation that resulted in identification of less than 20 human-specific proteins. In contrast to these earlier studies, we demonstrate that a more in-depth protein profiling strategy, such as the 4-D method, is crucial for successful identification of substantial numbers of human-specific proteins in xenograft mouse serum.

### Example 6

### Human-Specific Proteins Released by Ovarian Tumors

A group of interesting human proteins identified in this study are summarized in Table 2, and details of the peptide identifications are reported in Table 3. Interestingly, a substantial number of common, relatively abundant serum proteins, such as ITIH4, APOA1, TTR, and TF are shed by the ovarian tumors. Some of these common plasma proteins were observed in human ovarian tumor specimens in prior reports, but it was not clear whether these proteins infiltrated the tissue from the blood or if the tumor produced these proteins. Some of these abundant proteins also have been associated with host-response or acute-phase reaction to biological insults and are primarily synthesized by the liver. However, the identification of multiple, human-specific peptides for these proteins unambiguously demonstrates that they were produced and shed into the blood by the ovarian tumors.

Despite their unambiguous tumor origin, these proteins are of limited use as biomarkers for diagnosing or monitoring ovarian cancer in humans, because the contribution to blood levels from small tumors is swamped by shedding from other tissues and normal variations in the protein's level in the normal population. Interestingly, three of these proteins, APOA1, TTR and a fragment of ITIH4 used in a multimarker panel, were reported to have higher diagnostic accuracy than CA125 for detection of early-stage ovarian cancer.¹⁴ However, a subsequent study found that the use of these proteins in biomarker panels did not outperform CA125 when used in prediagnostic samples.¹⁸

Among the biomarkers identified for use in serum, are certain previously discussed proteins. For example, the CTSD level has been proposed as a prognostic factor in a variety of cancers, including ovarian cancer.^{50, 51} Recently, it also was shown that quantitation of circulating autoantibody against CTSD can differentiate benign ovarian conditions from ovarian carcinoma.⁵² Similarly, CLU was shown to be present at a higher level in serum of late-stage ovarian cancer patients versus normal controls.³⁵ A number of other proteins such as LDHB, CFL1, CLIC1, and AKR1B1 also were identified previously in ovarian cancer tissues or in conditioned media for ovarian cancer cell lines,^{35, 53} but they were not previously known to be shed into the blood.

Most of the human proteins identified have at least 70% sequence identity with their mouse counterpart, and some share ≧90% identity with the mouse protein. An example of a highly homologous protein is CLIC1, which shares 98% sequence identity between the two species. Despite this very high sequence homology, a human-specific peptide, LAALNPESNTAGLDIFAK (amino acids 96-113 of SEQ ID NO:1), was identified that indicated the protein was shed by the human ovarian tumor (see FIG. 4). These data demonstrate that very highly homologous human and mouse proteins can be distinguished using the xenograft mouse model and the 4-D serum protein profiling method.

The production by the ovarian tumors and shedding into the mouse blood of proteins previously associated with ovarian cancer argue that this model effectively mimics normal protein shedding of ovarian cancer in humans. In addition, the xenograft mouse model allows detection of low-abundance serum proteins such as CTSD, which has a reported serum concentration of about 16 ng/mL.^{54, 55} Another important benefit of the use of the xenograft mouse model over cell lines, tumor tissues, and secretomes from cells or tissues is the ability to show that the identified proteins are actually shed into blood.

The human proteins identified herein are both unambiguously produced by the ovarian tumor and shed into the blood, and anticipated to do so in ovarian cancer patients. Hence, they are biomarkers for ovarian cancer in human patients. As noted above, the most cancer-specific biomarkers are those proteins shed primarily by the tumor and not other organs such as the liver. Therefore, candidate biomarkers from the xenograft mouse model that either have not been reported previously in normal human serum or are known to be low abundance are high-priority candidates for screening in ovarian cancer patient sera.

### Example 7

### Verification of Selected Candidate Ovarian Cancer Biomarkers in Human Ovarian Cancer Patient Sera

The utility of several high-priority candidate biomarkers for screening ovarian cancer patients was evaluated in a small patient cohort, as outlined in FIG. 1A and described in Tang et al.⁴⁵ A subset of seven human proteins within the 20 to 55 kDa range was selected (CTSD, CLIC1, AKR1B1, HMX1, TRPM1, CUTA, and SERPINB12) for initial evaluation (Table 2). In addition, a higher abundant known ovarian-cancer-associated serum protein (CLU) was included as a positive control.

Although these human proteins were detected in the xenograft mouse serum, the ability to detect them in human serum using GeLC-MRM was unknown. Therefore, multiple peptides from each of these proteins initially were targeted by LC-MS/MS on an LTQ-Orbitrap XL mass spectrometer using a pool of abundant protein-depleted serum from nine late-stage ovarian cancer patients (Table 2). The targeted MS/MS analysis was able to identify multiple peptides for CLU, CTSD, and CLIC1. However, only a single peptide was identified for AKR1B1 and HMX1, and three proteins (TRPM1, CUTA, and SERPINB12) could not be identified. The inability to detect TRPM 1, CUTA, and SERPINB12 is most likely due to very low concentrations in human serum, as suggested by the identification of only a single peptide in the xenograft mouse system. Our minimum criteria for high-confidence GeLC-MRM quantitation required detection and quantitation of at least two peptides per protein and, therefore, AKR1B1 and HMX1 were not included in the MRM assays.

GeLC-MRM quantitation was performed initially on separate pools of nine serum samples from patients diagnosed with benign, and late- (stages III and IV) stage ovarian cancer. To assess the robustness of the MRM methods for the selected proteins and to obtain a preliminary indication of the predictive value of CLU, CTSD, and CLIC1, gel slices identified as containing the proteins in the xenograft mouse analysis, as well as adjacent fractions, were analyzed, and relative peptide amounts were summed across gel slices (FIGS. 5A-5C). As expected, typically all peptides from a given protein displayed similar trends across the three pooled serum samples. In the case of CLU, one peptide was disproportionately low in the benign samples. Examination of the raw data showed splitting of the peptide peak, apparently due to variations in spray in the triple quadrupole nanosource. Hence, this peptide was not used for protein quantitation in this dataset.

It should also be noted that some serum proteins such as CTSD and CLU undergo proteolytic processing to yield mature forms of the protein. Although both the full-length and mature forms are detectable in serum if all fractions of the gel are analyzed, it is more efficient if analysis focuses on a discrete region of the gel to maximize throughput. We focused on the 20-55 kDa region, which included the mature forms of CTSD and CLU, but not the full-length (unprocessed) forms of these proteins.

The results obtained from a preliminary analysis of the pooled samples showed that the CLIC1 and the CTSD mature forms (henceforth referred to as CTSD-30 kDa) exhibited the greatest difference between benign and late-stage ovarian cancer. The levels of these proteins were measured in individual control serum samples (six normal and nine benign), and late-stage cancer samples (15 stage III and 3 stage IV). We did not continue to evaluate CLU because it is a known high-abundant plasma protein with reported concentration ranging from 58 to 150 μg/ml,^{35, 56} and its level is also elevated by many acute-phase stimuli such as inflammation, heat shock, and injury.^{57, 58}

In addition to CLIC1 and CTSD-30 kDa, PRDX6 was included in subsequent analyses. PRDX6 is a 25 kDa bifunctional 1-Cys peroxiredoxin that has been hypothesized to promote cancer growth and invasiveness, with increased expression observed in some malignancies.⁵⁹⁻⁶¹ The mouse PRDX6 was identified with four peptides that are indistinguishable from human. In addition, PRDX6 was identified in a TOV-112D secretome study.³⁵ Taken together with our results, our results suggest PRDX6, which is in the 25 kDa region being assayed, is a biomarker for ovarian cancer.

Label-free MRM quantitation of individual serum samples showed a significant difference (P<0.05, Student's t-test) between the control (normal and benign) and cancer groups for CLIC1 and CTSD-30 kDa (FIGS. 6A, 6C and 6E, left panels). The normal and benign samples also were compared separately to the cancer group, since the normal and benign samples were collected at two different sites and benign conditions often are more difficult to distinguish from cancer than healthy controls (FIGS. 6B, 6D and 6F, right panels).

PRDX6 showed a significant difference between normal controls and cancer, but not between benign disease and cancer, whereas there were significant differences between cancer and either non-cancer group for the other two biomarkers.

To further evaluate the potential diagnostic efficacy for each of the three proteins, receiver operating characteristic curve analyses were performed on the control and cancer groups (FIGS. 7A-7C). In agreement with the t-test, both CLIC1 and CTSD-30 kDa showed a larger area under the ROC (AUC) compared to PRDX6. The sensitivity and specificity, as well as the positive and negative predictive values for each biomarker at the optimal cut-point, are presented in Table 5.

TABLE 5

| | | | | Positive | Negative |
|---|---|---|---|---|---|
| | | | | Predictive | Predictive |
| Biomarker | Cut-point^{b} | Sensitivity | Specificity | Value | Value |
| CTSD- | 0.378 | 88.9 | 73.3 | 80.0 | 84.6 |
| 30 kDa | | | | | |
| CLIC1 | 0.495 | 66.7 | 86.7 | 85.7 | 68.4 |
| PRDX6 | 0.517 | 66.7 | 80.0 | 80.0 | 66.7 |

^{a}Cases are 18 tumor samples and 15 normal/benign samples.
^{b}Optimal cut-points expressed as normalized relative abundance and defined by maximizing sensitivity and specificity.

A binary decision rule for CTSD1-30 kDa, CLIC1, and PRDX6 was created using their optimal cut-point. Each binary variable was a significant predictor of tumor samples (p=0.001. 0.005, and 0.011, respectively). In the multivariate analysis, only CTSD1-30 kDa and CLIC1 remained in the final model (p=0.009 and 0.052, respectively). The AUC for the predicted probability of a tumor sample from the multivariate model, including these two biomarkers, was 0.893.

To evaluate the reproducibility of the entire label-free GeLC-MRM^{45, 62} workflow for the ovarian cancer biomarkers, we prepared two separate serum pools of normal (n=6) and ovarian cancer (n=9) samples and subjected the two pooled samples to major protein depletion and GeLC-MRM quantitation, as shown in FIG. 1B. MRM quantitation of CTSD-30 kDa, CLIC1, and PRDX6 in this new pool of samples then was compared to the averaged quantitation values of individual samples that made up the pooled samples. As shown in FIG. 8, MRM quantitation of the normal and cancer samples is very similar for the pool samples and the average of individual samples, demonstrating the reproducibility of the entire label-free GeLC-MRM workflow. In this analysis, the level of CTSD did not appear to be significantly different between the two groups due to the inclusion of more low responders in the cancer group. This highlights the potential risks of using pooled serum samples to gauge the predictive value of a biomarker, and the importance of analyzing individual samples in biomarker validation.

These results indicate that CTSD-30 kDa and CLIC1 and PRDX6 are biomarkers of ovarian cancer. Similar test performed on Tropomyosin 1 (TPM1), proteasome subunit alpha type-7 (PSMA7) and bisphosphoglycerate mutase (BPGM) demonstrate that these are also biomarkers of ovarian cancer. See, e.g., FIGS. 9A-9D and 10A-10B.

To our knowledge, CTSD, CLIC1, and PRDX6 levels in serum of ovarian cancer patients have not been reported previously—although, a recent report did show that quantitation of circulating autoantibody against CTSD can differentiate between benign and other stages of ovarian carcinoma including stage I,⁵² which supports our argument that CTSD is a biomarker for ovarian cancer. Interestingly, CLIC1 recently was discovered as a novel plasma marker for nasopharyngeal carcinoma,⁶³ although its role in ovarian cancer is still unclear.

Each and every patent, patent application, and publication, including publications listed below, U.S. provisional patent application No. 61/532,881 and publically available peptide sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### PUBLICATIONS

1. Jemal, A. et al; Cancer statistics, 2010. CA Cancer J Clin 2010, 60, (5), 277-300.
2. Bell, D. A., Origins and molecular pathology of ovarian cancer. Mod Pathol 2005, 18 Suppl 2, S 19-32.
3. Kobel, M. et al; Ovarian carcinoma subtypes are different diseases: implications for biomarker studies. PLoS Med 2008, 5, (12), e232.
4. Cannistra, S. A., Cancer of the ovary. N Engl J Med 2004, 351, (24), 2519-29.
5. Mutch, D. G., Surgical management of ovarian cancer. Semin Oncol 2002, 29, (1 Suppl 1), 3-8.
6. Grover, S. R.; Quinn, M. A., Is there any value in bimanual pelvic examination as a screening test. Med J Aust 1995, 162, (8), 408-10.
7. Clarke-Pearson, D. L., Clinical practice. Screening for ovarian cancer. N Engl J Med 2009, 361, (2), 170-7.
8. Mutch, D. G., Ovarian cancer: to screen or not to screen. Obstet Gynecol 2009, 113, (4), 772-4.
9. Moss, E. L. et a; The role of CA125 in clinical practice. J Clin Pathol 2005, 58, (3), 308-12.
10. Nick, A. M.; Sood, A. K., The ROC ‘n’ role of the multiplex assay for early detection of ovarian cancer. Nat Clin Pract Oncol 2008, 5, (10), 568-9.
11. Terry, K. L. et al; Blood and urine markers for ovarian cancer: a comprehensive review. Dis Markers 2004, 20, (2), 53-70.
12. Dutta, S. et al; Biomarkers for ovarian cancer detection and therapy. Cancer Biol Ther 2010, 9, (9), 668-77.
13. Cramer, D. W. et al; Ovarian cancer biomarker performance in prostate, lung, colorectal, and ovarian cancer screening trial specimens. Cancer Prev Res (Phila) 2011, 4, (3), 365-74.
14. Zhang, Z.; et al., Three biomarkers identified from serum proteomic analysis for the detection of early stage ovarian cancer. Cancer Res 2004, 64, (16), 5882-90.
15. Zhang, Z. et al, Combining multiple serum tumor markers improves detection of stage I epithelial ovarian cancer. Gynecol Oncol 2007, 107, (3), 526-31.
16. Gorelik, E. et al, Multiplexed immunobead-based cytokine profiling for early detection of ovarian cancer. Cancer Epidemiol Biomarkers Prev 2005, 14, (4), 981-7.
17. Visintin, I. et al, Diagnostic markers for early detection of ovarian cancer. Clin Cancer Res 2008, 14, (4), 1065-72.
18. Zhu, C. S. et al, A framework for evaluating biomarkers for early detection: validation of biomarker panels for ovarian cancer. Cancer Prev Res (Phila) 2011, 4, (3), 375-83.
19. Pitteri, S.; Hanash, S., A systems approach to the proteomic identification of novel cancer biomarkers. Dis Markers 2010, 28, (4), 233-9.
20. Anderson, N. L.; Anderson, N. G., The human plasma proteome: history, character, and diagnostic prospects. Mol Cell Proteomics 2002, 1, (11), 845-67.
21. Rifai, N. et al., Protein biomarker discovery and validation: the long and uncertain path to clinical utility. Nat Biotechnol 2006, 24, (8), 971-83.
22. Tang, H. Y. et al, A novel four-dimensional strategy combining protein and peptide separation methods enables detection of low-abundance proteins in human plasma and serum proteomes. Proteomics 2005, 5, (13), 3329-42.
23. Tang, H. Y.; Speicher, D. W., Complex proteome prefractionation using microscale solution isoelectrofocusing. Expert Rev Proteomics 2005, 2, (3), 295-306.
24. Jacobs, J. M. et al, Utilizing human blood plasma for proteomic biomarker discovery. J Proteome Res 2005, 4, (4), 1073-85.
25. Lee, H. J. et al, Biomarker discovery from the plasma proteome using multidimensional fractionation proteomics. Curr Opin Chem Biol 2006, 10, (1), 42-9.
26. Zeng, Z. et al, A Proteomics Platform Combining Depletion, Multi-lectin Affinity Chromatography (M-LAC), and Isoelectric Focusing to Study the Breast Cancer Proteome. Anal Chem 2011, 83, (12), 4845-54.
27. Faca, V. et al, Contribution of protein fractionation to depth of analysis of the serum and plasma proteomes. J Proteome Res 2007, 6, (9), 3558-65.
28. Gagne, J. P. et al, Proteome profiling of human epithelial ovarian cancer cell line TOV-112D. Mol Cell Biochem 2005, 275, (1-2), 25-55.
29. Dai, L. et al, Comparative proteomic study of two closely related ovarian endometrioid adenocarcinoma cell lines using cIEF fractionation and pathway analysis. Electrophoresis 2009, 30, (7), 1119-31.
30. Zhu, Y. et al, Classifications of ovarian cancer tissues by proteomic patterns. Proteomics 2006, 6, (21), 5846-56.
31. Bengtsson, S. et al, Large-scale proteomics analysis of human ovarian cancer for biomarkers. J Proteome Res 2007, 6, (4), 1440-50.
32. Gortzak-Uzan, L. et al, A proteome resource of ovarian cancer ascites: integrated proteomic and bioinformatic analyses to identify putative biomarkers. J Proteome Res 2008, 7, (1), 339-51.
33. Kuk, C. et al, Mining the ovarian cancer ascites proteome for potential ovarian cancer biomarkers. Mol Cell Proteomics 2009, 8, (4), 661-9.
34. Faca, V. M. et al, Proteomic analysis of ovarian cancer cells reveals dynamic processes of protein secretion and shedding of extra-cellular domains. PLoS One 2008, 3, (6), e2425.
35. Gunawardana, C. G. et al, Comprehensive analysis of conditioned media from ovarian cancer cell lines identifies novel candidate markers of epithelial ovarian cancer. J Proteome Res 2009, 8, (10), 4705-13.
36. Wei, B. R. et al, Serum S100A6 concentration predicts peritoneal tumor burden in mice with epithelial ovarian cancer and is associated with advanced stage in patients. PLoS One 2009, 4, (10), e7670.
37. Pitteri, S. J. et al, Integrated proteomic analysis of human cancer cells and plasma from tumor bearing mice for ovarian cancer biomarker discovery. PLoS One 2009, 4, (11), e7916.
38. He, Y. et al, LC-MS/MS analysis of ovarian cancer metastasis-related proteins using a nude mouse model: 14-3-3 zeta as a candidate biomarker. J Proteome Res 2010, 9, (12), 6180-90.
39. Juan, H. F. et al, Identification of tumor-associated plasma biomarkers using proteomic techniques: from mouse to human. Proteomics 2004, 4, (9), 2766-75.
40. Dafou, D. et al, Chromosomes 6 and 18 induce neoplastic suppression in epithelial ovarian cancer cells. Int J Cancer 2009, 124, (5), 1037-44.
41. Zuo, X. et al, Towards global analysis of mammalian proteomes using sample prefractionation prior to narrow pH range two-dimensional gels and using one-dimensional gels for insoluble and large proteins. Electrophoresis 2001, 22, (9), 1603-15.
42. Tabb, D. L. et al, DTASelect and Contrast: tools for assembling and comparing protein identifications from shotgun proteomics. J Proteome Res 2002, 1, (1), 21-6.
43. Beer, L. A. et al, Plasma Biomarker Discovery Using 3D Protein Profiling Coupled with Label-Free Quantitation. Methods Mol Biol 2011, 728, 3-27.
44. Omenn, G. S. et al, Overview of the HUPO Plasma Proteome Project: results from the pilot phase with 35 collaborating laboratories and multiple analytical groups, generating a core dataset of 3020 proteins and a publicly-available database. Proteomics 2005, 5, (13), 3226-45.
45. Tang, H. Y. et al, Rapid Verification of Candidate Serological Biomarkers Using Gel-based, Label-free, Multiple Reaction Monitoring. J Proteome Res 2011, 10(9):4005-4017 (published online Jul. 26, 2011).
46. Provencher, D. M. et al, Characterization of four novel epithelial ovarian cancer cell lines. In Vitro Cell Dev Biol Anim 2000, 36, (6), 357-61.
47. Bijian, K. et al, Serum proteomic approach for the identification of serum biomarkers contributed by oral squamous cell carcinoma and host tissue microenvironment. J Proteome Res 2009, 8, (5), 2173-85.
48. Orazine, C. I. et al, A proteomic analysis of the plasma glycoproteins of a MCF-7 mouse xenograft: a model system for the detection of tumor markers. J Proteome Res 2008, 7, (4), 1542-54.
49. van den Bemd, G. J. et al, Mass spectrometric identification of human prostate cancer-derived proteins in serum of xenograft-bearing mice. Mol Cell Proteomics 2006, 5, (10), 1830-9.
50. Benes, P. et al, Cathepsin D—many functions of one aspartic protease. Crit. Rev Oncol Hematol 2008, 68, (1), 12-28.
51. Losch, A. et al, Cathepsin D in ovarian cancer: prognostic value and correlation with p53 expression and microvessel density. Gynecol Oncol 2004, 92, (2), 545-52.
52. Taylor, D. D. et al, Patient-derived tumor-reactive antibodies as diagnostic markers for ovarian cancer. Gynecol Oncol 2009, 115, (1), 112-20.
53. Saraswat, M. et al, Overexpression of aldose reductase in human cancer tissues. Med Sci Monit 2006, 12, (12), CR525-529.
54. Merseburger, A. S. et al, Cathepsin D serum levels are not a valid serum marker in renal cell carcinoma. Urol Int 2007, 79, (1), 41-3.
55. Fukuda, M. E. et al, Cathepsin D is a potential serum marker for poor prognosis in glioma patients. Cancer Res 2005, 65, (12), 5190-4.
56. Hortin, G. L. et al, High-abundance polypeptides of the human plasma proteome comprising the top 4 logs of polypeptide abundance. Clin Chem 2008, 54, (10), 1608-16.
57. Falgarone, G.; Chiocchia, G., Chapter 8: Clusterin: A multifacet protein at the crossroad of inflammation and autoimmunity. Adv Cancer Res 2009, 104, 139-70.
58. Trougakos, I. P.; Gonos, E. S., Chapter 9: Oxidative stress in malignant progression: The role of Clusterin, a sensitive cellular biosensor of free radicals. Adv Cancer Res 2009, 104, 171-210.
59. Manevich, Y.; Fisher, A. B., Peroxiredoxin 6, a 1-Cys peroxiredoxin, functions in antioxidant defense and lung phospholipid metabolism. Free Radic Biol Med 2005, 38, (11), 1422-32.
60. Lee, S. B. et al, Peroxiredoxin 6 promotes ovarian cancer cell invasion by inducing urokinase-type plasminogen activator via p38 kinase, phosphoinositide 3-kinase, and Akt. Mol Cells 2009, 28, (6), 583-8.
61. Ho, J. N. et al, Phospholipase A2 activity of peroxiredoxin 6 promotes invasion and metastasis of ovarian cancer cells. Mol Cancer Ther 2010, 9, (4), 825-32.
62. Beer, L. A. et al, Systematic discovery of ectopic pregnancy serum biomarkers using 3-D protein profiling coupled with label-free quantitation. J Proteome Res 2011, 10, (3), 1126-38.
63. Chang, Y. H. et al, Cell secretome analysis using hollow fiber culture system leads to the discovery of CLIC1 protein as a novel plasma marker for nasopharyngeal carcinoma. J Proteome Res 2009, 8, (12), 5465-74.
64. Kozak, K. R. et al, Characterization of serum biomarkers for detection of early stage ovarian cancer. Proteomics 2005, 5, (17), 4589-96.
65. Scheffer, G. L. et al, Increased expression of beta 2-microglobulin in multidrug-resistant tumour cells. Br J Cancer 2002, 86, (12), 1943-50.
66. Simaga, S. et al, Quantitative biochemical analysis of lactate dehydrogenase in human ovarian tissues: correlation with tumor grade. Int J Gynecol Cancer 2005, 15, (3), 438-44.
67. Vazquez, J. et al, Expression and clinical significance of apolipoprotein D in epithelial ovarian carcinomas. Gynecol Oncol 2000, 76, (3), 340-7.
68. Tang, H-Y. et al, A Xenograft Mouse Model coupled with In-depth Plasma Proteome Analysis Facilitates Identification of Novel Serum Biomarkers for human Ovarian Cancer, J. Proteome Res., 2012 11:678-691.

1. A diagnostic reagent, kit, panel or microarray comprising at least one ligand capable of specifically complexing with, binding to, or quantitatively detecting or identifying a single target biomarker selected from the group consisting of:
a. cathepsin D-30 kDa (CTSD-30)
b. chloride intracellular channel protein 1 (CLIC1)
c. peroxieredoxin-6 (PRDX6)
d. tropomyosin 1 (TPM1)
e. bisphosphoglycerate mutase (BPGM); and
f. proteasome subunit alpha type-7 (PSMA7);
g. aldose reductase (AKR1B1)
h. homeobox protein (HMX1)
i. melastatin 1 (TRPM1)
j. protein CutA (CUTA)
k. SERPINB12 protein (SERPINB12),
l. cathepsin D-52 kDa (CTSD-52), and
m. an isoform, pro-form, modified molecular form, or peptide fragment of any of biomarkers (a) through (l),
wherein at least one ligand is associated with a detectable label or immobilized on a substrate.

2. The reagent, kit, panel or microarray according to claim 1, comprising multiple ligands selected from (a) through (m), each ligand directed to a different biomarker.

3. (canceled)

4. The reagent, kit, panel or microarray according to claim 1, further comprising at least one ligand that specifically complexes with, binds to, quantitatively detects or identifies the biomarker, CA125, or an isoform, pro-form, modified molecular form, or peptide fragment therefrom.

5. The reagent, kit, panel or microarray according to claim 1, wherein said ligand is selected from an antibody or fragment of an antibody, antibody mimic or equivalent that binds to or complexes with a biomarker of (a) through (m).

6. The reagent, kit, panel or microarray according to claim 4, wherein one or more ligands are immobilized on a substrate, each ligand specifically complexing with, binding to, quantitatively detecting or identifying a different biomarker selected from (a) to (m).

7-9. (canceled)

10. The reagent, kit, panel or microarray according to claim 1 comprising a ligand that binds or complexes individually to an additional known marker, isoform, pro-form, modified molecular form, or peptide fragment.

11-12. (canceled)

13. A method for diagnosing or detecting or monitoring the progress of ovarian cancer in a subject comprising:
(a) contacting a sample obtained from a test subject with a composition of claim 1;
(b) detecting or measuring in the sample or from a protein level profile generated from the sample, the protein levels of one or more of the biomarkers (a) to (m), or ratios thereof;
(c) comparing the protein levels of the biomarker in the subject's sample or from a protein level profile or ratio of multiple said biomarkers, with the level of the same biomarker or biomarkers in a reference standard;
wherein a significant change in protein level of the subject's sample biomarker or biomarkers from that in the reference standard indicates a diagnosis, risk, or the status of progression or remission of ovarian cancer in the subject.

14. The method according to claim 13, wherein the reference standard is a mean, an average, a numerical mean or range of numerical means, a numerical pattern, a ratio, a graphical pattern or a protein level profile derived from the same biomarker or biomarkers in a reference subject or reference population.

15-16. (canceled)

17. The method according to claim 13, wherein the subject's sample has been provided at a time selected from the group consisting of:
(a) before any ovarian cancer diagnosis;
(b) after a diagnosis of ovarian cancer;
(c) following surgical removal of an ovarian tumor;
(d) prior to surgical removal of an ovarian tumor;
(e) periodically following therapeutic treatment for an ovarian tumor;
(f) periodically during therapeutic treatment for an ovarian cancer;
(g) prior to therapeutic treatment for an ovarian tumor; and
(h) before diagnosis but with clinical symptoms of abdominal paid or abdominal symptom of unknown origin.

18. The method according to claim 13, wherein said change in protein level of each said biomarker comprises an increase in comparison to said reference or control or a decrease in comparison to said reference or control.

19. (canceled)

20. The method according to claim 13, wherein said detecting involves monitoring relapse after initial diagnosis and treatment, or predicting clinical outcome; or determining the best clinical treatment.

21-22. (canceled)

23. The method according to claim 13, wherein the biological sample is selected from group consisting of whole blood, plasma, serum, circulating tumor cells, ascites fluid, peritoneal fluid, a biopsy sample, surgical sample, or tumor cell or tissue sample.

24. The method according to claim 23, comprising performing a serum/plasma sandwich ELISA, or performing a mass spectrometry-based test, or performing a MRM assay wherein antibodies are used to enrich the biomarker protein or one or more peptides produced by specific proteolysis in a manner analogous to the capture antibody in sandwich ELISAs.

25-26. (canceled)

27. The method according to claim 13, which is performed by a computer processor or computer-programmed instrument that generates numerical or graphical data useful in the diagnosis of the condition.

28. The method according to claim 13, wherein the biomarker or biomarkers are present in different levels or abundance profiles in biological samples of two or more of the conditions selected from:
(a) no ovarian cancer;
(b) benign ovarian nodules;
(c) a subtype of epithelial ovarian cancer
(d) following surgical removal of an ovarian tumor;
(e) prior to surgical removal of an ovarian tumor;
(f) following therapeutic treatment for an ovarian tumor;
(g) periodically during treatment for ovarian tumor;
(h) prior to therapeutic treatment for an ovarian tumor; and
(i) undiagnosed clinical symptoms of abdominal pain or other abdominal condition of unknown origin;
(j) early stage ovarian cancer;
(k) advanced stage ovarian cancer;
(l) ovarian sarcoma,
(m) serous ovarian cancer;
(n) mucinous ovarian cancer;
(o) clear cell ovarian cancer;
(p) endometrioid ovarian cancer;
(q) Mullerian ovarian cancer;
(r) undifferentiated ovarian cancer; and
(s) serous papillary adenocarcinoma.

29. The reagent, kit, panel or microarray according to claim 1 comprising a ligand which is a nucleotide sequence capable of hybridizing to a nucleic acid sequence encoding a biomarker of (a) through (m), said ligand associated with a detectable label or with a substrate.

30. A method of diagnosing, or detecting a risk of developing, an ovarian cancer in a subject comprising:
(a) contacting a sample obtained from a test subject with a composition of claim 29;
(b) detecting or measuring in the sample or from an expression profile generated from the sample, the expression levels of one or more of the biomarkers (a) to (m), or ratios thereof;
(c) comparing the expression levels of the biomarker in the subject's sample or from an expression level profile or ratio of multiple said biomarkers, with the level of the same biomarker or biomarkers in a reference standard;
wherein a significant change in expression level of the subject's sample biomarker or biomarkers from that in the reference standard indicates a diagnosis, risk, or the status of progression or remission of ovarian cancer in the subject.

31. (canceled)

32. A method for diagnosing or detecting or monitoring the progress of ovarian cancer in a subject comprising:
(a) fragmenting proteins in a sample obtained from a test subject after contact with a chemical or enzymatic agent;
(b) injecting the digested sample of (a) into a mass spectrometer and identifying the protein levels of one or more of the biomarkers (a) to (m) of claim 1, or ratios thereof, by mass spectrometry;
(c) comparing the protein levels of the biomarker in the subject's sample, with the level of the same biomarker or biomarkers in a reference standard;
wherein a significant change in protein level of the subject's sample biomarker or biomarkers from that in the reference standard or from a predetermined cutoff indicates a diagnosis, risk, or the status of progression or remission of ovarian cancer in the subject.

33. The method according to claim 32, further comprising enriching the biomarker protein or one or more peptides produced by specific proteolysis in the sample by contacting the sample with an antibody prior to injecting into a mass spectrometer or liquid chromatographic mass spectrometer.

34. The method according to claim 32, comprising depleting non-target proteins in the sample prior to injecting sample into a mass spectrometer.

35-36. (canceled)


SEQUENCE LISTING (88 sequences)

SEQ ID NO: 1 - protein, 241 aa, Homo sapiens
MAEEQPQVELFVKAGSDGAKIGNCPFSQRLFMVLWLKGVTFNVTTVDTKRRTETVQKLCPGGQLPFLLYGTEVHTDTNKIEEFLEAVLCPPRYPKLAALNPESNTAGLDIFAKFSAYIKNSNPALNDNLEKGLLKALKVLDNYLTSPLPEEVDETSAEDEGVSQRKFLDGNELTLADCNLLPKLHIVQVVCKKYRGFTIPEAFRGVHRYLSNAYAREEFASTCPDDEEIELAYEQVAKALK

SEQ ID NO: 2 - protein, 241 aa, Mus musculus
MAEEQPQVELFVKAGSDGAKIGNCPFSQRLFMVLWLKGVTFNVTTVDTKRRTETVQKLCPGGQLPFLLYGTEVHTDTNKIEEFLEAMLCPPRYPKLAALNPESNTSGLDIFAKFSAYIKNSNPALNDNLEKGLLKALKVLDNYLTSPLPEEVDETSAEDEGISQRKFLDGNELTLADCNLLPKLHIVQVVCKKYRGFTIPEAFRGVHRYLSNAYAREEFASTCPDDEEIELAYEQVARALK

SEQ ID NO: 3 - protein, 8 aa, Homo sapiens
QVFGEATK

SEQ ID NO: 4 - protein, 8 aa, Homo sapiens
VGFAEAAR

SEQ ID NO: 5 - protein, 8 aa, Homo sapiens
SGSGLVGR

SEQ ID NO: 6 - protein, 12 aa, Homo sapiens
ASSIIDELFQDR

SEQ ID NO: 7 - protein, 23 aa, Homo sapiens
VTTVASHTSDSDVPSGVTEVVVK

SEQ ID NO: 8 - protein, 12 aa, Homo sapiens
SLQEQADAAEER

SEQ ID NO: 9 - protein, 6 aa, Homo sapiens
YEEEIK

SEQ ID NO: 10 - protein, 19 aa, Homo sapiens
AVGPHQFLGDQEAIQAAIK

SEQ ID NO: 11 - protein, 10 aa, Homo sapiens
HYGALIGLNR

SEQ ID NO: 12 - protein, 8 aa, Homo sapiens
LLPYWNER

SEQ ID NO: 13 - protein, 15 aa, Homo sapiens
NYTDEAIETDDLTIK

SEQ ID NO: 14 - protein, 14 aa, Homo sapiens
LTVEDPVTVEYITR

SEQ ID NO: 15 - protein, 11 aa, Homo sapiens
ALLEVVQSGGK

SEQ ID NO: 16 - protein, 13 aa, Homo sapiens
KFDGILGMAYPRI

SEQ ID NO: 17 - protein, 13 aa, Unknown
  mouse peptide
KFDGILGMGYPHI

SEQ ID NO: 18 - protein, 19 aa, Homo sapiens
KLFDSDPITVTVPVEVSRK

SEQ ID NO: 19 - protein, 19 aa, Unknown
  mouse peptide
KLFDSDPITVVLPEEVSKD

SEQ ID NO: 20 - protein, 11 aa, Homo sapiens
KGSEMVVAGKL

SEQ ID NO: 21 - protein, 15 aa, Homo sapiens
KNGIDIYSLTVDSRV

SEQ ID NO: 22 - protein, 10 aa, Homo sapiens
KNVVFVIDKS

SEQ ID NO: 23 - protein, 18 aa, Homo sapiens
KSPEQQETVLDGNLIIRY

SEQ ID NO: 24 - protein, 11 aa, Homo sapiens
KYIFHNFMERL

SEQ ID NO: 25 - protein, 17 aa, Homo sapiens
RANTVQEATFQMELPKK

SEQ ID NO: 26 - protein, 11 aa, Homo sapiens
RFAHTVVTSRV

SEQ ID NO: 27 - protein, 12 aa, Homo sapiens
RFKPTLSQQQKS

SEQ ID NO: 28 - protein, 14 aa, Homo sapiens
RGPDVLTATVSGKL

SEQ ID NO: 29 - protein, 24 aa, Homo sapiens
RQGPVNLLSDPEQGVEVTGQYERE

SEQ ID NO: 30 - protein, 13 aa, Homo sapiens
KATEHLSTLSEKA

SEQ ID NO: 31 - protein, 13 aa, Homo sapiens
KDLATVYVDVLKD

SEQ ID NO: 32 - protein, 16 aa, Homo sapiens
KLLDNWDSVTSTFSKL

SEQ ID NO: 33 - protein, 9 aa, Homo sapiens
KPALEDLRQ

SEQ ID NO: 34 - protein, 12 aa, Homo sapiens
KVQPYLDDFQKK

SEQ ID NO: 35 - protein, 14 aa, Homo sapiens
KVSFLSALEEYTKK

SEQ ID NO: 36 - protein, 15 aa, Homo sapiens
RDYVSQFEGSALGKQ

SEQ ID NO: 37 - protein, 18 aa, Homo sapiens
REQLGPVTQEFWDNLEKE

SEQ ID NO: 38 - protein, 13 aa, Homo sapiens
RQGLLPVLESFKV

SEQ ID NO: 39 - protein, 13 aa, Homo sapiens
RTHLAPYSDELRQ

SEQ ID NO: 40 - protein, 12 aa, Homo sapiens
RVNHVTLSQPKI

SEQ ID NO: 41 - protein, 15 aa, Homo sapiens
KAADDTWEPFASGKT

SEQ ID NO: 42 - protein, 23 aa, Homo sapiens
KTSESELHGLTTEEEFVEGIYKV

SEQ ID NO: 43 - protein, 15 aa, Homo sapiens
RGSPAINVAVHVFRK

SEQ ID NO: 44 - protein, 24 aa, Homo sapiens
RYTIAALLSPYSYSTTAVVTNPKE

SEQ ID NO: 45 - protein, 19 aa, Homo sapiens
KEDLIWELLNQAQEHFGKD

SEQ ID NO: 46 - protein, 16 aa, Homo sapiens
KEDPQTFYYAVAVVKK

SEQ ID NO: 47 - protein, 13 aa, Homo sapiens
KHSTIFENLANKA

SEQ ID NO: 48 - protein, 13 aa, Homo sapiens
KSASDLTWDNLKG

SEQ ID NO: 49 - protein, 14 aa, Homo sapiens
RDQYELLCLDNTRK

SEQ ID NO: 50 - protein, 10 aa, Homo sapiens
KFIIPQIVKY

SEQ ID NO: 51 - protein, 11 aa, Homo sapiens
KIVVVTAGVRQ

SEQ ID NO: 52 - protein, 18 aa, Homo sapiens
KLIAPVAEEEATVPNNKI

SEQ ID NO: 53 - protein, 12 aa, Homo sapiens
KSADTLWDIQKD

SEQ ID NO: 54 - protein, 17 aa, Homo sapiens
KSLADELALVDVLEDKL

SEQ ID NO: 55 - protein, 11 aa, Homo sapiens
RGLTSVINQKL

SEQ ID NO: 56 - protein, 14 aa, Homo sapiens
RVIGSGCNLDSARF

SEQ ID NO: 57 - protein, 12 aa, Homo sapiens
KAVLFCLSEDKK

SEQ ID NO: 58 - protein, 13 aa, Homo sapiens
KAVLFCLSEDKKN

SEQ ID NO: 59 - protein, 22 aa, Homo sapiens
KEILVGDVGQTVDDPYATFVKM

SEQ ID NO: 60 - protein, 14 aa, Homo sapiens
KHELQANCYEEVKD

SEQ ID NO: 61 - protein, 16 aa, Homo sapiens
KHELQANCYEEVKDRC

SEQ ID NO: 62 - protein, 19 aa, Homo sapiens
KKEDLVFIFWAPESAPLKS

SEQ ID NO: 63 - protein, 14 aa, Homo sapiens
KLGGSAVISLEGKP

SEQ ID NO: 64 - protein, 12 aa, Homo sapiens
KTLLSNLEEAKK

SEQ ID NO: 65 - protein, 13 aa, Homo sapiens
KTLLSNLEEAKKK

SEQ ID NO: 66 - protein, 14 aa, Homo sapiens
RASSIIDELFQDRF

SEQ ID NO: 67 - protein, 13 aa, Homo sapiens
RELDESLQVAERL

SEQ ID NO: 68 - protein, 13 aa, Homo sapiens
RKTLLSNLEEAKK

SEQ ID NO: 69 - protein, 25 aa, Homo sapiens
RVTTVASHTSDSDVPSGVTEVVVKL

SEQ ID NO: 70 - protein, 16 aa, Homo sapiens
KCPNPPVQENFDVNKY

SEQ ID NO: 71 - protein, 12 aa, Homo sapiens
KNLTSNNIDVKK

SEQ ID NO: 72 - protein, 15 aa, Homo sapiens
RNPNLPPETVDSLKN

SEQ ID NO: 73 - protein, 20 aa, Homo sapiens
KAIGAVPLIQGEYMIPCEKV

SEQ ID NO: 74 - protein, 20 aa, Homo sapiens
RDPDAQPGGELMLGGTDSKY

SEQ ID NO: 75 - protein, 19 aa, Homo sapiens
RISVNNVLPVFDNLMQQKL

SEQ ID NO: 76 - protein, 19 aa, Homo sapiens
KFLDGNELTLADCNLLPKL

SEQ ID NO: 77 - protein, 14 aa, Homo sapiens
KGVTFNVTTVDTKR

SEQ ID NO: 78 - protein, 20 aa, Homo sapiens
KLAALNPESNTAGLDIFAKF

SEQ ID NO: 79 - protein, 14 aa, Homo sapiens
KNSNPALNDNLEKG

SEQ ID NO: 80 - protein, 10 aa, Homo sapiens
RLFMVLWLKG

SEQ ID NO: 81 - protein, 32 aa, Homo sapiens
KGIVVTAYSPLGSPDRPWAKPEDPSLLEDPRI

SEQ ID NO: 82 - protein, 12 aa, Homo sapiens
KMPILGLGTWKS

SEQ ID NO: 83 - protein, 9 aa, Homo sapiens
KTTQVLIRF

SEQ ID NO: 84 - protein, 20 aa, Homo sapiens
KYKPAVNQIECHPYLTQEKL

SEQ ID NO: 85 - protein, 19 aa, Homo sapiens
RSGAAGGSGAGGAWPGGRH

SEQ ID NO: 86 - protein, 17 aa, Homo sapiens
KAQSHQLFAIIMECMKK

SEQ ID NO: 87 - protein, 29 aa, Homo sapiens
RSVHPYEVAEVIALPVEQGNFPYLQWVRQ

SEQ ID NO: 88 - protein, 12 aa, Homo sapiens
KTDYTLSIANRL